(19) United States
(12) Patent Application Publication
(10) Pub. No.: US 2009/0325999 A1
(43) Pub. Date: Dec. 31, 2009

(54) PERSONALIZED PHARMACEUTICAL KITS, PACKAGING AND COMPOSITIONS FOR THE TREATMENT OF ALLERGIC CONDITIONS

(76) Inventor: Jie Du, Lansdale, PA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 12/147,810

(22) Filed: Jun. 27, 2008

(51) Int. Cl. A61K 31/437; A61K 31/135; A61K 31/165

(52) U.S. Cl. 514/299; 514/651; 514/618

(57) ABSTRACT

This invention relates to personalized pharmaceutical kits, packaging, compositions, and methods for treatment of a mammal, comprising at least one antihistamine for treating an allergic disease or condition in a mammal, in combination with at least one wakefulness promoting agent for preventing sedative effects during day time use, while promoting the antihistamine and sedating effect during evening use.

### BACKGROUND OF THE INVENTION

1. Field of Invention

This invention relates to personalized pharmaceutical kits, packaging, compositions, and methods for treatment of a mammal, comprising a combination of active pharmaceutical ingredients, for example an antihistamine for treating an allergic disease or condition in a mammal, in combination with, for example, a wakefulness promoting agent for preventing sedative effects during day time use, while promoting the antihistamine and sedating effect during evening use.

2. Description of Related Art

Histamine is a biologically active amine found in many tissues and is frequently released locally to induce complex physiologic and pathologic effects through multiple histamine receptor subtypes. Three different receptor sites for histamine have been recognized, and are designated H1, H2, and H3. Histamine is recognized as an important mediator of immediate allergic and inflammatory reactions. Generally, upon exposure of the body to a variety of immunogenic stimuli, such as pollen, dust, toxins, and the like, histamine is released into the blood circulation to induce an allergic-type response in the body. Particularly, histamine stored in mast cells and basophil cells is often released upon sensitization by IgE antibodies attached to the cell surface membranes. Histamine released by this mechanism is a mediator in immediate (type 1) allergic reactions. In addition, by a negative feedback control mechanism mediated by H2 receptors, histamine appears to modulate its own release and that of other mediators from sensitized mast cells in some tissues. In addition to inducing allergic response, histamine plays many other roles, including a role in acute inflammatory responses.

Histamine is also known to exert powerful effects on smooth and cardiac muscle, on certain endothelia and nerve cells, and on secretory cells of the stomach. Particularly, histamine causes contraction of intestinal smooth muscle. Large doses of histamine may even cause diarrhea. Histamine has long been recognized as a powerful stimulant of gastric acid secretion and, to a lesser extent, of gastric pepsin and intrinsic factor production. In the central nervous system, histamine stimulates sensory nerve endings, especially those mediating pain and itching. This H1 mediated effect is an important component of the urticarial response and reactions to insect and nettle stings. Pre-synaptic H3 receptors play important roles in modulating neurotransmitter release from peripheral nerve endings. In the cardiovascular system, histamine causes a decrease in systolic and diastolic blood pressure and an increase in heart rate. Particularly, the acute blood pressure changes are caused by the direct vasodilator action of histamine on arterial and capillary sphincters and the increase in heart rate involves both stimulatory actions of histamine on the heart, and a reflex tachycardia. Histamine induced edema results from the action of the histamine on the H1 receptors in the blood vessels. The effect is associated with the separation of endothelial cells, which permits the transudation of fluid and molecules as large as small proteins into the perivascular tissue. This effect is responsible for the urticaria (hives) that signals the release of histamine in the skin.

Histamine also causes bronchial constriction mediated by H1 receptors. Thus, humans suffering from asthma are very sensitive to histamine. The bronchial constriction induced in these patients probably represents a hyperactive neural response, since such patients also respond excessively to many other stimuli, and their response to histamine can be blocked by autonomic blocking drugs such as ganglionic blocking agents as well as H1 receptor antagonists. Sensitivity to histamine, however, varies greatly among mammals.

Thus, the release of histamine in the body causes many adverse physiological and pathological responses or effects in mammals and is induced, or stimulated, by common, everyday environmental factors and/or allergies to those factors. There is, therefore, a need to reduce or alleviate these effects and/or prevent their induction by histamine.

The effects of histamine released in the body may be treated or addressed in several ways. Histamine receptor antagonists, such as epinephrine, are generally referred to as antihistamines and administered to counter the effects of histamine. But epinephrine antagonists, and derivatives thereof, act at receptors other than those activated by histamine and are, therefore, not highly effective in alleviating or preventing a histamine-mediated response. Other antihistamines, such as diphenhydramine, act directly on the histamine receptor(s) to reduce or prevent histamine-mediated responses. For example, medications containing diphenhydramine, or similarly structured compounds, are available to competitively antagonize many of the actions of histamine.

Over the years, a number of antihistamines (histamine antagonists) have been developed. Many antihistamines have relatively strong sedative effects. Many antihistamines are capable of crossing the blood-brain barrier and entering the central nervous system, and have a sedating effect. Common antihistamines include compounds such as diphenhydramine, brompheniramine, hydrazine, and chloropheniramine, all of which are able to cross the blood-brain barrier and are sedating in effect. The intensity of sedation varies among the antihistamines depending upon the chemical structure. For some antihistamines, the sedation is so prominent that they are useful as “sleep aids” and unsuitable for daytime use. Sedation, however, also renders these antihistamines potentially dangerous depending upon the person's activity after taking the antihistamine. For example, driving after having taken a recommended dose of an antihistamine may result in falling asleep at the wheel consequentially presenting risks of injury, property damage, etc. Accordingly, sedation may interfere with everyday activity and is, therefore, typically an undesirable effect of the use of antihistamines.

There are 3 generations of antihistamine products:
1^{st} generation: OTC products with severe sedating side effect, such as diphenhydramine, chloropheniramine, etc. They have been established as the most effective antihistamines with the highest safety profiles. However due to the sedating side effect, they are not widely used.
2^{nd} generation: Non sedating compounds with cardiovascular toxicity (causing QT prolongation). They are prescription products. Most of them have been pulled off market due to safety issues, such as terfenadine (Seldane®).
3^{rd} generation: Rx non-sedating products with good safety profile, such as Claritin®, Allegra®. They are very widely advertised through television programs (DTC advertising). However their efficacy is low. Very often, these “non-sedating” drugs do cause sedation (contrary to commercial advertisement), and they are not effective in relieving allergy symptoms. There have been numerous lawsuits again the manufacturers' misleading efficacy claims of these products. See the link below on Claritin® lawsuit at www.pubmedcentral.nih.gov/articlerender.fcgi?artid=1121020

The prior art (US patent publication: US2004/0259809A1) described a combination of sedating antihistamines and stimulants (such as amphetamines) to create a non-sedating antihistamine allergy treatment. However, when this approach was tried on patients, it was disappointed to find out that the stimulant present in the product working very well during daytime actually caused an annoying side effect that patients could not fall asleep at night time. See treatment D under Example 5. Therefore, there is a need to provide pharmaceutical kits, methods, packaging, and compositions, comprising an antihistamine, which may be administered in a therapeutically effective dose, in combination with, for example, a wakefulness promoting agent, thus minimizing undesirable side effects, such as sedation for daytime use. While within the same product of this invention, for evening use, both the antihistamine effectiveness and sedating side effects are promoted. This invention focuses (but not limit to) on the 1st generation antihistamines as mentioned above.

Accordingly, it is an object of the present invention to provide an effective treatment for an allergic disease or condition by providing a combination of medications particularly adapted for administration with regard to time of day and/or a particular event and organized via packaging and/or indicia for use together in a treatment regimen. Among the benefits achieved are improving the patient's therapy, and avoiding unwanted side effects inherent in taking the same medication for all activities and events, and avoiding the risks of taking a multiplicity of uncoordinated medications; while promoting the commonly considered sedating side effect of a pharmaceutical at a different time of a day as one of the unique attributes of the pharmaceutical kit/package.

The present invention provides a combination of separately formulated dosage units which together are effective for the treatment of allergies, the dosage units being formulated so as to be adapted for administration during separate events or times of day. The present invention provides a pharmaceutical housing that contains a combination of at least two dosage units within one product, each of which is effective for the treatment of an allergic disease or condition, one dosage unit being adapted for administration during one time of day or for a particular event, and the other dosage unit being adapted for administration during a different time of day or for a different event. The housing and/or the dosage units have indicia for distinguishing the different types of dosage units from each other, and the housing contains dosing instructions for administration of the different types of dosage units together in an allergy treatment regimen.

The invention also provides antihistamine containing pharmaceutical compositions with removed sedative effects by including a wakefulness promoting agent in the composition. The present invention addresses the weaknesses and drawbacks of prior art antihistamine-containing compositions by providing unique pharmaceutical compositions that may be administered in effective dosages for treating histamine-mediated symptoms with a removed sedative effect by targeting certain part of a day. To this end, the pharmaceutical compositions, kits, packages, and dosage forms, for example, include at least one antihistamine and at least one wakefulness promoting agent. The most potent antihistamines are generally sedating in nature and the sedation is alleviated with the wakefulness promoting agent. The pharmaceutical compositions, kits, and methods are useful for treating, for example, allergic reactions and other histamine-mediated symptoms, as well as for providing other physiological effects including, for example, anticholinergic effects, analgesic effects, analgesic adjuvant effects, soporific effects, anti-secretory effects, and combination effects thereof. By combining a potent, effective antihistamine with an effective, anti-sedating wakefulness promoting agent, the compositions of the present invention can be administered more safely and effectively than prior art antihistamine-containing medications utilized for the same purpose, since this combination targets only certain part of a day. The present invention also provides methods of use for the pharmaceutical compositions and kits. There exists a need in the art for a combination of a sedating antihistamine with wakefulness promoting agent in dosage forms and/or packaging for a daily dosage regimen without the problems associated with most wakefulness promoting agent administration (i.e. increased heart rate, jumpiness, anxiety, etc.).

All references cited herein are incorporated herein by reference in their entireties.

### BRIEF SUMMARY OF THE INVENTION

The invention provides a personalized pharmaceutical packaging system for administering a plurality of pharmaceutical active ingredients to an individual in need thereof according to a personalized daily regimen, said personalized pharmaceutical packaging system comprising: a therapeutic dosage to be administered in the AM, said therapeutic dosage comprising first active ingredient of at least one antihistamine, or a pharmaceutically acceptable salt or derivative thereof, and second active ingredient of at least one wakefulness promoting agent, or a pharmaceutically acceptable salt or derivative thereof, wherein the first and second active ingredients are in multiple separated dosage units, or in a single dosage unit comprising a combination of active ingredients, optionally, a therapeutic dosage to be administered in the midday, comprising a first active ingredient of at least one antihistamine, or a pharmaceutically acceptable salt or derivative thereof, and an optional second active ingredient of at least one wakefulness promoting agent, or a pharmaceutically acceptable salt or derivative thereof, wherein the first and second active ingredients are in multiple separated dosage units, or in a single dosage unit comprising a combination of active ingredients, a therapeutic dosage to be administered in the PM or bedtime, comprising at least one unit dosage comprising at least one antihistamine, or a pharmaceutically acceptable salt or derivative thereof, a means for containing said unit dosage or dosages, wherein said means for containing said unit dosage or dosages further comprises: (i) at least one printable surface; and/or (ii) a memory aid for administering said unit dosage or dosages; and/or (iii) directions for administering the unit dosage or dosages, and optionally wherein the personalized dosage regimen is customized for the needs of the individual based on criteria relevant to the individuals personal information.

The invention further provides a personalized pharmaceutical packaging system, wherein the personal information comprises any one or combination of the following: the ethnicity of the individual, the sex of the individual, the weight of the individual, the Body Mass Index of the individual, age of the individual, the incidence of a condition of potential interest for the personalized therapeutic regimen in the individual's family, the environmental conditions of the individual, and combinations thereof.

The invention further provides a personalized pharmaceutical packaging system, wherein the personalized dosage regimen is based on the body weight of the individual, and is selected from the group consisting of: an AM dosage for an individual less than 100 lb, comprising a range of about 6-50 mg diphenhydramine and a wakefulness promoting agent selected from the group consisting of about 50-150 mgmodafinil, about 25-100 mgarmodafinil, and about 50-150 mg Caffeine per dosage unit, an AM dosage for an individual of 95-170 lb, comprising a range of about 25-75 mg diphenhydramine and a wakefulness promoting agent selected from the group consisting of about 100-200 mg modafinil, about 75-150 mg armodafinil, and about 130-240 mg Caffeine per dosage unit, an AM dosage for an individual of 150-300 lb, comprising a range of about 50-100 mg diphenhydramine and a wakefulness promoting agent selected from the group consisting of about 150-250 mg modafinil, 125-200 mg armodafinil, and about 220-340 mg Caffeine per dosage unit, an AM dosage for an individual over 270 lb, comprising a range of about 75-125 mg diphenhydramine and a wakefulness promoting agent selected from the group consisting of about 200-350 mg modafinil, about 150-225 mg armodafinil, and about 310-425 mg Caffeine per dosage unit, a PM dosage for an individual less than 100 lb, comprising a range of about 6-50 mg diphenhydramine per dosage unit, a PM dosage for an individual of 95-170 lb, comprising a range of about 25-75 mg diphenhydramine per dosage unit, a PM dosage for an individual of 150-300 lb, comprising a range of about 50-100 mg diphenhydramine per dosage unit, and a PM dosage for an individual over 270 lb, comprising a range of about 75-125 mg diphenhydramine per dosage unit.

The invention further provides a personalized pharmaceutical packaging system, wherein the means for containing said AM, PM or MIDDAY therapeutic dosage or dosages is selected from the group consisting of: the first and second unit dosages are packaged together in a single package or packette, the first and second unit dosages are packaged separately in a plurality of packages or packettes; a blister packet; a lidded blister; or blister card or packets, a shrink wrap, and with both drugs released upon opening of the single package or packette, a plurality of packages or packettes; blister packet; lidded blister or blister card or packets; or shrink wrap; a blister pack; a container; and a device, and further wherein the AM, PM and optional MIDDAY dosages are separated from each other within the personalized pharmaceutical packaging system.

The invention further provides a personalized pharmaceutical packaging system wherein said system is selected from the group consisting of: a connected bottle pack comprising an AM bottle, a PM bottle, and an optional MIDDAY bottle; and a blister package comprising: a) a rupturable substrate b) a layer forming one or more blisters over the rupturable substrate, wherein each of the one or more blisters contain one or more unit dosage forms.

The invention provides a method of making a personalized pharmaceutical packaging system for administering a plurality of pharmaceutical active ingredients to an individual in need thereof according to a personalized daily regimen, said personalized pharmaceutical packaging system comprising: a therapeutic dosage to be administered in the AM, said therapeutic dosage comprising first active ingredient of at least one antihistamine, or a pharmaceutically acceptable salt or derivative thereof, and second active ingredient of at least one wakefulness promoting agent, or a pharmaceutically acceptable salt or derivative thereof, wherein the first and second active ingredients are in multiple separated dosage units, or in a single dosage unit comprising a combination of active ingredients; optionally, a therapeutic dosage to be administered in the midday, comprising a first active ingredient of at least one antihistamine, or a pharmaceutically acceptable salt or derivative thereof, and an optional second active ingredient of at least one wakefulness promoting agent, or a pharmaceutically acceptable salt or derivative thereof, wherein the first and second active ingredients are in multiple separated dosage units, or in a single dosage unit comprising a combination of active ingredients; a therapeutic dosage to be administered in the PM or bedtime, comprising at least one unit dosage comprising at least one antihistamine, or a pharmaceutically acceptable salt or derivative thereof; a means for containing said unit dosage or dosages, wherein said means for containing said unit dosage or dosages further comprises: (i) at least one printable surface; and/or (ii) a memory aid for administering said unit dosage or dosages; and/or (iii) directions for administering the unit dosage or dosages; and optionally wherein the personalized dosage regimen is customized for the needs of the individual based on criteria relevant to the individuals personal information. The invention further provides a method of making a personalized pharmaceutical packaging system, wherein the personal information comprises any one or combination of the following: the ethnicity of the individual; the sex of the individual; the weight of the individual; the Body Mass Index of the individual; age of the individual; the incidence of a condition of potential interest for the personalized therapeutic regimen in the individual's family; the environmental conditions of the individual; and combinations thereof.

The invention further provides a method of making a personalized pharmaceutical packaging system, wherein the personalized dosage regimen is based on the body weight of the individual, and is selected from the group consisting of: an AM dosage for an individual less than 100 lb, comprising a range of about 6-50 mg diphenhydramine and a wakefulness promoting agent selected from the group consisting of about 50-150 mg modafinil, about 25-100 mg armodafinil, and about 50-150 mg Caffeine per dosage unit; an AM dosage for an individual of 95-170 lb, comprising a range of about 25-75 mg diphenhydramine and a wakefulness promoting agent selected from the group consisting of about 100-200 mg modafinil, about 75-150 mg armodafinil, and about 130-240 mg Caffeine per dosage unit; an AM dosage for an individual of 150-300 lb, comprising a range of about 50-100 mg diphenhydramine and a wakefulness promoting agent selected from the group consisting of about 150-250 mg modafinil, 125-200 mg armodafinil, and about 220-340 mg Caffeine per dosage unit; an AM dosage for an individual over 270 lb, comprising a range of about 75-125 mg diphenhydramine and a wakefulness promoting agent selected from the group consisting of about 200-350 mg modafinil, about 150-225 mg armodafinil, and about 310-425 mg Caffeine per dosage unit; a PM dosage for an individual less than 100 lb, comprising a range of about 6-50 mg diphenhydramine per dosage unit; a PM dosage for an individual of 95-170 lb, comprising a range of about 25-75 mg diphenhydramine per dosage unit; a PM dosage for an individual of 150-300 lb, comprising a range of about 50-100 mg diphenhydramine per dosage unit; and a PM dosage for an individual over 270 lb, comprising a range of about 75-125 mg diphenhydramine per dosage unit.

The invention further provides a method of making a personalized pharmaceutical packaging system, wherein the means for containing said AM, PM or MIDDAY therapeutic dosage or dosages is selected from the group consisting of: the first and second unit dosages are packaged together in a single package or packette; the first and second unit dosages are packaged separately in a plurality of packages or packettes; a blister packet; a lidded blister; or blister card or packets; a shrink wrap, and with both drugs released upon opening of the single package or packette; a plurality of packages or packettes; blister packet; lidded blister or blister card or packets; or shrink wrap; a blister pack; a container; and a device, and further wherein the AM, PM and optional MIDDAY dosages are separated from each other within the personalized pharmaceutical packaging system.

The invention further provides a method of making a personalized pharmaceutical packaging system wherein said system is selected from the group consisting of: a connected bottle pack comprising an AM bottle, a PM bottle, and an optional MIDDAY bottle; and a blister package comprising: a) a rupturable substrate b) a layer forming one or more blisters over the rupturable substrate, wherein each of the one or more blisters contain one or more unit dosage forms.

The invention provides a method of treating a disease or condition in mammal by using a personalized pharmaceutical packaging system for administering a plurality of pharmaceutical active ingredients to an individual in need thereof according to a personalized daily regimen, said personalized pharmaceutical packaging system comprising: a therapeutic dosage to be administered in the AM, said therapeutic dosage comprising first active ingredient of at least one antihistamine, or a pharmaceutically acceptable salt or derivative thereof, and second active ingredient of at least one wakefulness promoting agent, or a pharmaceutically acceptable salt or derivative thereof, wherein the first and second active ingredients are in multiple separated dosage units, or in a single dosage unit comprising a combination of active ingredients; optionally, a therapeutic dosage to be administered in the midday, comprising a first active ingredient of at least one antihistamine, or a pharmaceutically acceptable salt or derivative thereof, and an optional second active ingredient of at least one wakefulness promoting agent, or a pharmaceutically acceptable salt or derivative thereof, wherein the first and second active ingredients are in multiple separated dosage units, or in a single dosage unit comprising a combination of active ingredients; a therapeutic dosage to be administered in the PM or bedtime, comprising at least one unit dosage comprising at least one antihistamine, or a pharmaceutically acceptable salt or derivative thereof; a means for containing said unit dosage or dosages, wherein said means for containing said unit dosage or dosages further comprises: (i) at least one printable surface; and/or (ii) a memory aid for administering said unit dosage or dosages; and/or (iii) directions for administering the unit dosage or dosages; and optionally wherein the personalized dosage regimen is customized for the needs of the individual based on criteria relevant to the individuals personal information.

The invention further provides a method of treating a disease or condition in mammal by using a personalized pharmaceutical packaging system, wherein the personal information comprises any one or combination of the following: the ethnicity of the individual; the sex of the individual; the weight of the individual; the Body Mass Index of the individual; age of the individual; the incidence of a condition of potential interest for the personalized therapeutic regimen in the individual's family; the environmental conditions of the individual; and combinations thereof.

The invention further provides a method of treating a disease or condition in mammal by using a personalized pharmaceutical packaging system, wherein the personalized dosage regimen is based on the body weight of the individual, and is selected from the group consisting of: an AM dosage for an individual less than 100 lb, comprising a range of about 6-50 mg diphenhydramine and a wakefulness promoting agent selected from the group consisting of about 50-150 mg modafinil, about 25-100 mg armodafinil, and about 50-150 mg Caffeine per dosage unit; an AM dosage for an individual of 95-170 lb, comprising a range of about 25-75 mg diphenhydramine and a wakefulness promoting agent selected from the group consisting of about 100-200 mg modafinil, about 75-150 mg armodafinil, and about 130-240 mg Caffeine per dosage unit; an AM dosage for an individual of 150-300 lb, comprising a range of about 50-100 mg diphenhydramine and a wakefulness promoting agent selected from the group consisting of about 150-250 mg modafinil, 125-200 mg armodafinil, and about 220-340 mg Caffeine per dosage unit; an AM dosage for an individual over 270 lb, comprising a range of about 75-125 mg diphenhydramine and a wakefulness promoting agent selected from the group consisting of about 200-350 mg modafinil, about 150-225 mg armodafinil, and about 310-425 mg Caffeine per dosage unit; a PM dosage for an individual less than 100 lb, comprising a range of about 6-50 mg diphenhydramine per dosage unit; a PM dosage for an individual of 95-170 lb, comprising a range of about 25-75 mg diphenhydramine per dosage unit; a PM dosage for an individual of 150-300 lb, comprising a range of about 50-100 mg diphenhydramine per dosage unit; and a PM dosage for an individual over 270 lb, comprising a range of about 75-125 mg diphenhydramine per dosage unit.

The invention further provides a method of treating a disease or condition in mammal by using a personalized pharmaceutical packaging system, wherein the means for containing said AM, PM or MIDDAY therapeutic dosage or dosages is selected from the group consisting of: the first and second unit dosages are packaged together in a single package or packette; the first and second unit dosages are packaged separately in a plurality of packages or packettes; a blister packet; a lidded blister; or blister card or packets; a shrink wrap, and with both drugs released upon opening of the single package or packette; a plurality of packages or packettes; blister packet; lidded blister or blister card or packets; or shrink wrap; a blister pack; a container; and a device, and further wherein the AM, PM and optional MIDDAY dosages are separated from each other within the personalized pharmaceutical packaging system.

The invention further provides a method of treating a disease or condition in mammal by using a personalized pharmaceutical packaging system, wherein said system is selected from the group consisting of: a connected bottle pack comprising an AM bottle, a PM bottle, and an optional MIDDAY bottle; and a blister package comprising: a) a rupturable substrate b) a layer forming one or more blisters over the rupturable substrate, wherein each of the one or more blisters contain one or more unit dosage forms.

The invention provides a pharmaceutical unit dosage form according to any one of claims 3, 8, 13 wherein the dosage is selected from the group consisting of: an AM dosage for an individual less than 100 lb, comprising a range of about 6-50 mg diphenhydramine, and a wakefulness promoting agent selected from the group consisting of about 50-150 mg modafinil, about 25-100 mg armodafinil, and about 50-150 mg Caffeine per dosage unit; an AM dosage for an individual of 95-170 lb, comprising a range of about 25-75 mg diphenhydramine a wakefulness promoting agent selected from the group consisting of about 100-200 mg modafinil, about 75-150 mg armodafinil, and about 130-240 mg Caffeine per dosage unit; an AM dosage for an individual of 150-300 lb, comprising a range of about 50-100 mg diphenhydramine and a wakefulness promoting agent selected from the group consisting of about 150-250 mg modafinil, about 125-200 mg armodafinil, and about 220-340 mg Caffeine per dosage unit; an AM dosage for an individual over 270 lb, comprising a range of about 75-125 mg diphenhydramine and a wakefulness promoting agent selected from the group consisting of about 200-350 mg modafinil, about 150-225 mg armodafinil, and about 310-425 mg Caffeine per dosage unit; a PM dosage for an individual less than 100 lb, comprising a range of about 6-50 mg diphenhydramine per dosage unit; a PM dosage for an individual of 95-170 lb, comprising a range of about 25-75 mg diphenhydramine per dosage unit; a PM dosage for an individual of 150-300 lb, comprising a range of about 50-100 mg diphenhydramine per dosage unit; and a PM dosage for an individual over 270 lb, comprising a range of about 75-125 mg diphenhydramine per dosage unit.

The invention further provides a pharmaceutical unit dosage form, wherein the dosage form contains release-retarding material; wherein the release-retarding material is a release-retarding matrix, a release-retarding coating, or a combination comprising at least one of the foregoing; wherein the controlled-release formulation provides therapeutically effective plasma levels for greater than about 12 hours after administration at steady state; wherein the release-retarding material is selected from the group consisting of polyethylene glycols, waxes, cellulose derivatives, polyacrylate derivatives, polyvinylpyrrolidine, hydrogenated oils, shellac, zein, fatty acids, gums, and the like.

The invention further provides a pharmaceutical unit dosage form, wherein the pharmaceutical unit dosage is an oral dosage form comprising an antihistamine, or a pharmaceutically acceptable salt or derivative thereof, and an optional wakefulness promoting agent, or a pharmaceutically acceptable salt or derivative thereof.

The invention further provides a pharmaceutical unit dosage form, wherein the dosage form is a controlled-release pharmaceutical formulation comprising an antihistamine, or a pharmaceutically acceptable salt or derivative thereof, and a wakefulness promoting agent, or a pharmaceutically acceptable salt or derivative thereof; wherein said controlled release formulation exhibits an in vitro dissolution profile in simulated intestinal fluid medium comprising less than about 70% drug release after 1 hour, at least about 20% drug release after 4 hours, and at least about 30% drug release after 6 hours.

The invention further provides a pharmaceutical unit dosage form which comprises a controlled release component of a wakefulness promoting agent, or a pharmaceutically acceptable salt or derivative thereof and a pharmaceutically acceptable excipient; and an immediately release component of an antihistamine, or a pharmaceutically acceptable salt or derivative thereof; wherein said controlled release component exhibits an in vitro dissolution profile in simulated intestinal fluid medium comprising less than about 70% wakefulness promoting agent release after 1 hour, at least about 20% wakefulness promoting agent release after 4 hours, and at least about 30% wakefulness promoting agent release after 6 hours.

The invention further provides a pharmaceutical unit dosage form which comprises a controlled release component of an antihistamine, or a pharmaceutically acceptable salt or derivative thereof and a pharmaceutically acceptable excipient; and an immediately release component of a wakefulness promoting agent, or a pharmaceutically acceptable salt or derivative thereof; wherein said controlled release component exhibits an in vitro dissolution profile in simulated intestinal fluid medium comprising less than about 70% antihistamine release after 1 hour, at least about 20% antihistamine release after 4 hours, and at least about 30% antihistamine release after 6 hours.

The invention further provides a personalized pharmaceutical packaging system, wherein the means for containing said AM therapeutic dosage or dosages is selected from the group consisting of: the first and second unit dosages are packaged together in a single package or packette; the first and second unit dosages are packaged separately in a plurality of packages or packettes; a blister packet; a lidded blister; or blister card or packets; a shrink wrap, and with both drugs released upon opening of the single package or packette; a plurality of packages or packettes; blister packet; lidded blister or blister card or packets; or shrink wrap; a blister pack; a container; and a device, and further wherein the AM dosage is separated from the midday and PM therapeutic dosages within the personalized pharmaceutical packaging system.

The invention further provides a personalized pharmaceutical packaging system, wherein the means for containing the midday therapeutic dosage is selected from the group consisting of: the first and second unit dosages are packaged together in a single package or packette; the first and second unit dosages are packaged separately in a plurality of packages or packettes; a blister packet; a lidded blister or blister card or packets; shrink wrap, and with both drugs released upon opening of the single package or packette; a plurality of packages or packettes; blister packet; lidded blister or blister card or packets; or shrink wrap; a blister pack, a container; and a device, and further wherein the midday therapeutic dosage is separated from the AM and PM therapeutic dosages within the personalized pharmaceutical packaging system.

The invention further provides a personalized pharmaceutical packaging system, wherein the means for containing the PM therapeutic dosage or dosages is selected from the group consisting of: the first and second unit dosages are packaged together in a single package or packette; the first and second unit dosages are packaged separately in a plurality of packages or packettes; a blister packet; a lidded blister or blister card or packets; a shrink wrap, and with both drugs released upon opening of the single package or packette; a plurality of packages or packettes; blister packet; lidded blister or blister card or packets; or shrink wrap; a blister pack, a container; and a device, and further wherein the PM dosage is separated from the AM and midday therapeutic dosages within the personalized pharmaceutical packaging system.

The invention further provides a personalized pharmaceutical packaging system wherein said system is configured as a blister package comprising: a) a rupturable substrate b) a layer forming one or more blisters over the rupturable substrate; wherein each of the one or more blisters contain one or more unit dosage forms.

The invention further provides a method of making a personalized pharmaceutical packaging system, wherein the means for containing said AM therapeutic dosage or dosages is selected from the group consisting of: the first and second unit dosages are packaged together in a single package or packette; the first and second unit dosages are packaged separately in a plurality of packages or packettes; a blister packet; a lidded blister; or blister card or packets; a shrink wrap, and with both drugs released upon opening of the single package or packette; a plurality of packages or packettes; blister packet; lidded blister or blister card or packets; or shrink wrap; a blister pack; a container; and a device, and further wherein the AM dosage is separated from the midday and PM therapeutic dosages within the personalized pharmaceutical packaging system.

The invention further provides a method of making a personalized pharmaceutical packaging system, wherein the means for containing the midday therapeutic dosage is selected from the group consisting of: the first and second unit dosages are packaged together in a single package or packette; the first and second unit dosages are packaged separately in a plurality of packages or packettes; a blister packet; a lidded blister or blister card or packets; shrink wrap, and with both drugs released upon opening of the single package or packette; a plurality of packages or packettes; blister packet; lidded blister or blister card or packets; or shrink wrap; a blister pack; a container; and a device, and further wherein the midday therapeutic dosage is separated from the AM and PM therapeutic dosages within the personalized pharmaceutical packaging system.

The invention further provides a method of making a personalized pharmaceutical packaging system, wherein the means for containing the PM therapeutic dosage or dosages is selected from the group consisting of: the first and second unit dosages are packaged together in a single package or packette; the first and second unit dosages are packaged separately in a plurality of packages or packettes; a blister packet; a lidded blister or blister card or packets; a shrink wrap, and with both drugs released upon opening of the single package or packette; a plurality of packages or packettes; blister packet; lidded blister or blister card or packets; or shrink wrap; a blister pack; a container; and a device, and further wherein the PM dosage is separated from the AM and midday therapeutic dosages within the personalized pharmaceutical packaging system.

The invention further provides a method of making a personalized pharmaceutical packaging system, wherein said system is configured as a blister package comprising: a) a rupturable substrate b) a layer forming one or more blisters over the rupturable substrate; wherein each of the one or more blisters contain one or more unit dosage forms.

The invention further provides a method of treating a disease or condition, wherein the means for containing said AM therapeutic dosage or dosages is selected from the group consisting of: the first and second unit dosages are packaged together in a single package or packette; the first and second unit dosages are packaged separately in a plurality of packages or packettes; a blister packet; a lidded blister; or blister card or packets; a shrink wrap, and with both drugs released upon opening of the single package or packette; a plurality of packages or packettes; blister packet; lidded blister or blister card or packets; or shrink wrap; a blister pack; a container; and a device, and further wherein the AM dosage is separated from the midday and PM therapeutic dosages within the personalized pharmaceutical packaging system.

The invention further provides a method of treating a disease or condition, wherein the means for containing the midday therapeutic dosage is selected from the group consisting of: the first and second unit dosages are packaged together in a single package or packette; the first and second unit dosages are packaged separately in a plurality of packages or packettes; a blister packet; a lidded blister or blister card or packets; shrink wrap, and with both drugs released upon opening of the single package or packette; a plurality of packages or packettes; blister packet; lidded blister or blister card or packets; or shrink wrap; a blister pack, a container; and a device, and further wherein the midday therapeutic dosage is separated from the AM and PM therapeutic dosages within the personalized pharmaceutical packaging system.

The invention further provides a method of treating a disease or condition, wherein the means for containing the PM therapeutic dosage or dosages is selected from the group consisting of: the first and second unit dosages are packaged together in a single package or packette; the first and second unit dosages are packaged separately in a plurality of packages or packettes; a blister packet; a lidded blister or blister card or packets; a shrink wrap, and with both drugs released upon opening of the single package or packette; a plurality of packages or packettes; blister packet; lidded blister or blister card or packets; or shrink wrap; a blister pack; a container; and or a device, and further wherein the PM dosage is separated from the AM and midday therapeutic dosages within the personalized pharmaceutical packaging system.

The invention further provides a method of treating a disease or condition, wherein the system is configured as a blister package comprising: a) a rupturable substrate b) a layer forming one or more blisters over the rupturable substrate; wherein each of the one or more blisters contain one or more unit dosage forms.

### BRIEF DESCRIPTION OF SEVERAL VIEWS OF THE DRAWINGS

FIG. 1 is a graph of the average plasma concentration-time curves for diphenhydramine following oral administration of an extended release dosage form of a diphenhydramine/caffeine combination.

FIG. 2 is a graph of the average plasma concentration-time curves for diphenhydramine following oral administration of a 50 mg immediate release dosage form of diphenhydramine.

### DETAILED DESCRIPTION OF PREFERRED EMBODIMENTS OF THE INVENTION

A pharmaceutical product or Kit for effectively treating allergy symptoms contains an AM or daytime dosage and a PM or night time dosage. The pharmaceutical AM (or daytime) compositions, and methods, of the kits (products) of the present invention comprise at least one antihistamine and at least one wakefulness promoting agent and may be administered in a pharmaceutically acceptable formulation or extended release formulation. The antihistamine is therapeutically effective to treat histamine-mediated responses, such as by providing relief from upper-respiratory congestion and viral and allergic rhinitis. Generally, the most potent antihistamines are sedative in nature by crossing the blood-brain barrier. The wakefulness promoting agent is provided, therefore, to alleviate such sedation. Thus, the wakefulness promoting agent addresses potential undesirable side effects typically caused by many sedating antihistamines. For example, the daytime dosage of present invention would reduce or eliminate dangers related to driving while sedated, and related consequences such as personal injury, property damage, etc; while the pharmaceutical PM (or night time) composition and methods of the kits of the present invention comprise the sedating antihistamine to promote its sedating side effect and its antihistamine effect.

The present invention provides unique pharmaceutical compositions, and methods of use thereof, for treating histamine-mediated responses, particularly allergy and allergy-related symptoms, and providing beneficial physiological effects in a mammal, more safely than comparable medications of the prior art. Histamine-mediated responses refer to physiological responses or effects triggered, and often mediated, by the production, release, and binding of histamine to histamine-receptors in the body. Treatable histamine-mediated responses include a wide range of symptoms, including allergy symptoms related to a common cold or flu, such as congestion in the upper airways, cough, fever, runny nose, and the like, as well as hives, breakouts, itching, and swelling due to common allergens, such as pollen, dust, foods, and the like, or other external stimulants. The term “amount sufficient”, as used here, is intended to refer to an amount, of the ingredient to which it refers, capable of producing the physiological response for which the ingredient is known. For example, an amount sufficient of an antihistamine is an amount of the antihistamine capable of antagonizing the effects of histamine in the body (an antihistaminic effect).

The term “pharmaceutically acceptable salt”, as used herein with reference to an antihistamine and a wakefulness promoting agent, is intended to refer to all salt forms of an active ingredient, such as the antihistamine and wakefulness promoting agent. For example, any acid, base, or halide counter-ion which forms a salt with the active and is biologically safe for mammalian consumption is suitable. Non-limiting examples include a hydrochloride, a citrate, a maleate, a fumarate, a succinate, a saccarate, an aspartate, a sulfate, an amine salt, an amino acid salt, and the like. Pharmaceutically acceptable salts of active ingredients are generally known to those of ordinary skill in the art.

The term “derivative” as used herein, is intended to refer to compounds having the active chemical core structure with one or more inert structural modifications thereon. In addition, a derivative would include all such compounds, which do not change or alter the pharmacological mechanism of action and/or window of therapeutic efficacy of the core structure, including polymorphs, solvates, isomers, hydrates, and etc. For example, a “derivative” of diphenhydramine would include compounds having one or more methyl substitutions on the phenyl ring or aliphatic regions of the diphenhydramine core and which would not significantly differ in efficacy and mode of action. Many “derivative” compounds, for the antihistamine actives and wakefulness promoting agent actives, are known in the art and/or will be discovered and are contemplated herein. As such, the term “derivative” is used broadly herein, as appreciated by one of ordinary skill in the art.

### Antihistamines

Suitable antihistamines for use in the compositions, methods, and kits of the present invention include, without limitation, diphenhydramine, cyproheptadine hydrochloride, brompheneramine, hydroxyzine, chloropheniramine, pyrilamine maleate, pyrilamine tannate, acepromazine, aceprometazine, alimemazine, alimemazine tartrate, amoxydramine camsilate, antazoline chlorhydrate, antazoline mesilate, antazoline phosphate, astemizole, azatadine dimaleate, azelastine hydrochloride, epinastine, bamipine hydrochloride, benactyzine hydrochloride, bretylium tosilate, bromazine hydrochloride, brompheniramine maleate, buclizine dihydrochloride, bufexamac, carbinoxamine maleate acid, cetiedil citrate, cetirizine dihydrochloride, chlorcyclizine hydrochloride, chlorphenamine maleate, chlorphenoxamine hydrochloride, chlorprothixene hydrochloride, cinnarizine, clemastine fumarate, clemizole hexachlorophenate, clemizole penicilline, clemizole undecylenate, clocinizine dihydrochloride, clofedanol, clofenetamine hydrochloride, cyclizine hydrochloride, dexchlorpheniramine maleate, di(acefylline)diphenhydramine, difencloxazine, dimelazine hydrochloride, dimenhydrinate, dimethoxanate hydrochloride, cimetotiazine mesilate, diphenhydramine hydrochloride, diphenhydramine mesilate, diphenylpyraline hydrochloride, diproqualone camsilate, dixyrazine, doxylamine succinate, eprozinol dihydrochloride, etodroxizine dimaleate, etybenzatropine bromhydrate, etybenzatropine hydrochloride, etymemazine hydrochloride, fenethazine hydrochloride, fenoxazoline hydrochloride, fenpentadiol, flunarizine hydrochloride, flupentixol decanoate, flupentixol dihydrochloride, histapyrrodine hydrochloride, hydroxyzine dihydrochloride, hydroxyzine embonate, indoramine hydrochloride, isothipendyl hydrochloride, ketotifene fumarate, levocabastine hydrochloride, levomepromazine, levomepromazine hydrochloride, levomepromazine embonate, levomepromazine maleate, loratadine, maprotiline hydrochloride, maproti line mesi late, maprotiline resinate, meclozine hydrochloride, mecysteine hydrochloride, medifoxamine fumarate, mefenidramium metilsulfate, mepyramine maleate, mequitazine, methaqualone, methdilazine hydrochloride, metixene hydrochloride, mizolastine, moxisylyte hydrochloride, niaprazine, orphenadrine hydrochloride, oxaflumazine disuccinate, oxatomide, oxolamine benzilate, oxolamine citrate, oxomemazine, oxomemazine hydrochloride, parathiazine teoclate, perimetazine, pheniramine maleate, phenoxybenzamine hydrochloride, phenyltoloxamine, phenyltoloxamine citrate, pimethixene, pipotiazine, pipretecol dihydrochloride, pizotifene malate, prednazoline, profenamine hydrochloride, promethazine, promethazine hydrochloride, promethazine embonate, promethazine polyvinylbenzene-metacrylate, propiomazine, terfenadine, fexofenadine, thenalidine tartrate, thenyldiamine hydrochloride, thiazinamium metilsulfate, tripelennamine hydrochloride, triprolidine hydrochloride, tymazoline hydrochloride, and pharmaceutically acceptable salts or derivatives thereof. Further, combinations of the above exemplary antihistamines may be included to provide the desired antihistaminic effects.

Many of the antihistamines, or pharmaceutically acceptable salts or derivatives thereof, above provide advantages and therapeutic benefits in addition to their proven antihistaminic effects. Thus, a composition including such an antihistamine, or a pharmaceutical acceptable salt or derivative thereof, may be effective for alleviating multiple allergy and cold-type symptoms, as well as for providing other effects, in both children and adults. Accordingly, the AM/daytime composition of the present invention provides a modified and improved drug release profile, and improved therapeutic profile with reduced/removed side effects, comparing to prior art antihistamine therapies.

The antihistamine, or pharmaceutically acceptable salts or derivatives thereof, should be included in an amount sufficient to effectively alleviate the histamine-mediated response, such as an allergy symptom or allergy-related symptom, and/or to provide other physiological effects, in a single dose of the composition. The potency of the antihistamine is generally dependent upon the particular antihistamine or combination thereof. For example, diphenhydramine is known to be effective in amounts as low as about 6 mg per dose or greater, depending upon the patient. More specifically, a diphenhydramine dose of about 6 mg to a child may be effective. Accordingly, the effective dosage may vary, as appreciated by one of ordinary skill in the art, depending upon many factors, such as age, weight, and gender of the patient, as well as prior medical history and present medical health.

In an exemplary, non-limiting embodiment, the antihistamine, or a pharmaceutically acceptable salt or derivative thereof, is provided in a weight ranging from about 1 mg to about 500 mg per dose of the composition. In another embodiment, the antihistamine is present in a weight ranging from about 5 mg to about 400 mg per dose, and the composition is to be administered to an adult. In yet another embodiment, the composition includes antihistamine in a weight ranging from about 6 mg to about 200 mg, per dose, for administration to a child.

In an exemplary, non-limiting embodiment, the antihistamine is diphenhydramine, or a pharmaceutically acceptable salt or derivative thereof, in a weight ranging from about 6 mg to about 150 mg per dose of the composition. In another embodiment, diphenhydramine is present in a weight ranging from about 25 mg to about 100 mg per dose, and the composition is to be administered to an adult. In yet another embodiment, the composition includes diphenhydramine in a weight ranging from about 6.25 mg to about 25 mg, per dose, for administration to a child.

### Wakefulness Promoting Agent

The present invention provides pharmaceutical compositions, kits, and methods of use, for treating histamine-mediated responses, particularly allergy and allergy-related symptoms, and providing beneficial physiological effects in a mammal, more safely than comparable medications of the prior art by the inclusion of at least one wakefulness promoting agent during part of the treatment. Wakefulness promoting agents are drugs that principally act as or are used as a central nervous system activator. Useful in the practice of the invention are wakefulness promoting agents that operate on the sleep-wake centers of the brain and that lack the pharmacological effects of amphetamines. Wakefulness promoting agents have, for example, the pharmacological profile of modafinil. Thus, in an embodiment of the invention, the wakefulness promoting agent used in the practice of the invention is Caffeine, theophylline, Provigil® (modafinil), Nuvigil® (armodafinil), Adrafinil and their pharmaceutical salts, isomers, polymorphs, solvates and etc. Non-limiting examples ofwakefulness promoting drugs, include, e.g., amphetamine, amphetamine homologues, caffeine, cathinone, cocaine, ephedrine, methamphetamine, methylphenidate, modafinil, pemoline, phenmetrazine, one amphetamine or a pharmaceutically acceptable salt or derivative thereof, or combination of dextroamphetamine saccarate, amphetamine aspartate, dextroamphetamine sulfate, and/or amphetamine sulfate, at least one of dextroamphetamine, pemoline, methylphenidate, modafinil, armodafinil, adrafinil caffeine, theophylline and pharmaceutically acceptable salts or derivatives thereof, and combinations thereof, as well as additional active agents for treating other conditions, diseases, or disorders, are known to those of ordinary skill in the art.

An effective amount of the wakefulness promoting agent of the invention may be administered to a subject animal (typically a human but other animals, e.g., farm animals, pets and racing animals, can also be treated). An effective amount is an amount that prevents, inhibits, or terminates excessive sleepiness associated with, for example, administration of antihistamine, narcolepsy, obstructive sleep apnea/hypopnea syndrome, jet lag or wakefulness disturbances as a consequence ofjet lag, or other disorders (including diseases of the nervous system), e.g., hypersomnia, REM behavior disorder, frontal nocturnal dystonia, restless legs syndrome, insomnia, parasomnia, nocturnal epileptic seizure, nocturnal movement disorder, sleep-related diagnostic dilemma, sleep apnea associated with neurological disorders, shift worker sleep disorder (SWSD), Kleine-Levin syndrome, sleep/wake disorders in blind subjects, and Parkinsonism. An effective amount is an amount that also prevents, inhibits, or terminates residual sedation, fatigue, drowsiness, and lack of energy experienced as the result of, for example, the administration of antihistamine, anesthesia, or an amount that prevents, inhibits, or terminates sleep disturbances induced by neurological injuries, abnormalities, lesions, or surgery.

Examples of wakefulness promoting agents include, but are not limited to: modafinil, armodafinil, adrafinil, amphetamine (racemic), d-amphetamine, 1-amphetamine and d-amphetamine phosphate, amphetamine and d-amphetamine sulfate, amphetamine and d-amphetamine hydrochloride, amphetamine and d-amphetamine saccharate, and amphetamine and d-amphetamine aspartate, Caffeine, theophylline, amphetaminil, bemegride, benzphetamine, benzphetamine hydrochloride, brucine, chlorphentermine, clofenciclan, clortermine, deanol acetamidobenzoate, demanyl phosphate, dexoxadrol, diethpropion, doxapram hydrochloride, N-ethylamphetamine, ethamivan, etifelmin, etryptamine, fencamfamine, fenethylline, fenosolone, fenfluramine, flurothyl, hexacyclonate sodium, homocamfin, mazindol, megexamide, methamphetamine, nicotinic agonists, nikethamide, pemoline, pentylenetetrazole, phenidimetrazine, phendimetrazine tartrate, phenmetrazine, phenmetrazine hydrochloride, phentermine, picrotoxin, pipradrol, pipradrol hydrochloride, prolintane, pyrovalerone, racephedrine, racephedrine hydrochloride, tetrahydrobenzothienopyridines, pharmaceutically acceptable salts or derivatives thereof, and combinations thereof.

Modafinil (C15-H15-NO2-S), 2-(benzhydrylsulfinyl)acetamide, or 2-[(diphenylmethyl)sulfinyl]acetamide, is a synthetic acetamide derivative with wake-promoting activity, the structure of which has been described in French Patent No. 78 05 510 and in U.S. Pat. No. 4,177,290. Modafinil was tested in combination with various agents including apomorphine, amphetamine, reserpine, oxotremorine, hypnotics, yohimbine, 5-hydroxytryptophan, monoamine oxidase inhibitor (I.M.A.O.), and in several behavioral conditions, as described in the cited patents. The conclusion from such tests is that modafinil presents a neuropsychopharmacological spectrum characterized by the presence of excitation with hyperactivity and hypermotility; and by the absence of stereotypy except in strong doses, and as potentiating the effects of apomorphine and amphetamine. A method of preparation of a racemic mixture is described in the '290 patent and a method of preparation of a levorotatory isomer is described in U.S. Pat. No. 4,927,855 (both incorporated herein by reference). The levorotatory isomer is reported to be useful for treatment of hypersomnia, depression, Alzheimer's disease and to have activity towards the symptoms of dementia and loss of memory, especially in the elderly.

NUVIGIL® (armodafinil) is a wakefulness-promoting agent for oral administration. Armodafinil is the R-enantiomer of modafinil which is a mixture of the R— and Senantiomers. The chemical name for armodafinil is 2-[(R)-(diphenylmethyl)sulfinyl]acetamide. The molecular formula is C15H15NO2S and the molecular weight is 273.35. Armodafinil has appeared in U.S. Pat. Nos. 4,927,855; 7,132,570; 7,297,346; RE37516.

Adrafinil, modafinil and armodafinil are agents with activity in the central nervous system, and have been developed as a treatment for excessive daytime sleepiness associated with narcolepsy. The primary pharmacological activity of adrafinil, modafinil and armodafinil, like amphetamine-like agents, is to promote wakefulness. Adrafinil, Modafinil and armodafinil promote wakefulness in rats (Touret, et al., Neuroscience Letters, 189:43-46 (1995); Edgar and Seidel, J. Pharmacol. Exp. Ther., 283:757-69 (1997)), cats (Lin et al., Brain Research, 591:3 19-326 (1992)), canines (Shelton et al., Sleep 18(10):817-826, (1995)) and non-human primates (DS-93-023, pp 180-181; Hernant et al., Psychopharmacology, 103:28-32 (1991)), as well as in models mimicking clinical situations, such as sleep apnea (English bulldog sleep disordered breathing model) (Panckeri et al, 1996) and narcolepsy (narcoleptic canine) (Shelton et al., Sleep 18(10):817-826, (1995)). Adrafinil, Modafinil and armodafinil have also been demonstrated to be a useful agent in the treatment of Parkinson's disease (U.S. Pat. No. 5,180,745); in the protection of cerebral tissue from ischemia (U.S. Pat. No. 5,391,576); in the treatment of urinary and fecal incontinence (U.S. Pat. No. 5,401,776); and in the treatment of sleep apneas of central origin (U.S. Pat. No. 5,612,378). U.S. Pat. No. 5,618,845 describes modafinil preparations of a defined particle size less than about 200 microns that is more potent and safer than preparations containing a substantial proportion of larger particles.

### Salts and Racemates

Any of the compounds, antihistamines, and/or wakefulness promoting agents, and active agents described herein may be administered in the form of a salt, ester, amide, prodrug, active metabolite, conjugate, derivative, polymorphs, solvates, or the like, provided that the salt, ester, amide, prodrug, metabolite, conjugate, or other derivative is suitable pharmacologically, i.e., effective in the present method. Salts, esters, amides, prodrugs, conjugates, and other derivatives of the active agents may be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992). For example, acid addition salts may be prepared from a free base (e.g., a compound containing a primary amino group) using conventional methodology involving reaction of the free base with an acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Conversely, preparation of basic salts of any acidic moieties that may be present may be carried out in a similar manner using a pharmaceutically acceptable base, such as sodium hydroxide, potassium hydroxide, ammonium hydroxide, calcium hydroxide, trimethylamine, or the like. Preparation of esters involves reaction of a hydroxyl group with an esterification reagent, such as an acid chloride, or esterification of a free carboxylic acid group. Amides may be prepared from esters using suitable amine reactants, or they may be prepared from an anhydride or an acid chloride by reaction with ammonia or a lower alkyl amine. Prodrugs, conjugates, and active metabolites may also be prepared using techniques known to those skilled in the art or described in the pertinent literature. Prodrugs and conjugates are typically prepared by covalent attachment of a moiety that results in a compound that is therapeutically inactive until modified by an individual's metabolic system.

In addition, many of the active agents contain chiral centers and can thus be in the form of a single isomer or a racemic mixture of isomers. Chiral active agents may be in isomerically pure form, or they may be administered as a racemic mixture of isomers.

Other derivatives and analogs of the active agents may be prepared using standard techniques known to those skilled in the art of synthetic organic chemistry, or may be deduced by reference to the pertinent literature, such as but not limited to: hydrates, polymorphs, solvates, etc.

### Methods of Treatment

The present invention provides methods for treating an allergic disease or condition in a mammal, for example, a wakefulness promoting agent and an antihistamine combined together into a single unit dose. The invention provides, for example, a wakefulness promoting agent and an antihistamine that can also be administered separately as two or more distinct doses. Thus, in some embodiments of the invention, a method for treating an allergic disease or condition in a mammal can be through the use of combination dosage forms. In some embodiments of the invention a method for treating an allergic disease or condition in a mammal can be through the use of separate dosage forms—one or more wakefulness promoting agent doses and one or more antihistamine doses. Accordingly, a dose of a wakefulness promoting agent can be administered at a different time relative to the antihistamine dose or simultaneously (i.e., wakefulness promoting agent dose administration within less than 1 hour before or after administration of the antihistamine). However, if simultaneous administration is desired, the administration of the wakefulness promoting agent and antihistamine can also be through the use of a single unit dose including both a wakefulness promoting agent and an antihistamine.

In embodiments where the wakefulness promoting agent and antihistamine are in separate dosage forms, the administration of the wakefulness promoting agent can preferably occur within moments, or in less than 1 hour, or less than 5 hours, or less than 24 hours or less than 48 hours, or less than 72 hours before or after administration of the antihistamine, unless otherwise indicated by a particular method of treatment below.

### Dosage Forms

The compositions of the present invention can be processed by agglomeration, air suspension chilling, air suspension drying, balling, coacervation, coating, comminution, compression, cryopelletization, encapsulation, extrusion, wet granulation, dry granulation, homogenization, inclusion complexation, lyophilization, melting, microencapsulation, mixing, molding, pan coating, solvent dehydration, sonication, spheronization, spray chilling, spray congealing, spray drying, or other processes known in the art. The compositions can be provided in the form of a minicapsule, a capsule, a tablet, an implant, a troche, a lozenge (minitablet), a temporary or permanent suspension, an ovule, a suppository, a wafer, a chewable tablet, a quick or fast dissolving tablet, an effervescent tablet, a buccal or sublingual solid, a granule, a film, a sprinkle, a pellet, a bead, a pill, a powder, a triturate, a platelet, a strip or a sachet. Compositions can also be administered as a “dry syrup”, where the finished dosage form is placed directly on the tongue and swallowed or followed with a drink or beverage. These forms are well known in the art and are packaged appropriately. The compositions can be formulated for oral, nasal, buccal, ocular, urethral, transmucosal, vaginal, topical or rectal delivery.

The pharmaceutical composition can be coated with one or more enteric coatings, seal coatings, film coatings, barrier coatings, compress coatings, fast disintegrating coatings, or enzyme degradable coatings. Multiple coatings can be applied for desired performance. Further, the dosage form can be designed for immediate release, pulsatile release, controlled release, extended release, delayed release, targeted release, synchronized release, or targeted delayed release. For release/absorption control, solid carriers can be made of various component types and levels or thicknesses of coats, with or without an active ingredient. Such diverse solid carriers can be blended in a dosage form to achieve a desired performance. The definitions of these terms are known to those skilled in the art. In addition, the dosage form release profile can be affected by a polymeric matrix composition, a coated matrix composition, a multiparticulate composition, a coated multiparticulate composition, an ion-exchange resin-based composition, an osmosis-based composition, or a biodegradable polymeric composition. Without wishing to be bound by theory, it is believed that the release may be effected through favorable diffusion, dissolution, erosion, ion-exchange, osmosis or combinations thereof.

When formulated as a capsule, the capsule can be a hard or soft gelatin capsule, a starch capsule, or a cellulosic capsule. Although not limited to capsules, such dosage forms can further be coated with, for example, a seal coating, an enteric coating, an extended release coating, or a targeted delayed release coating. These various coatings are known in the art, but for clarity, the following brief descriptions are provided: seal coating, or coating with isolation layers: Thin layers of up to 20 microns in thickness can be applied for variety of reasons, including for particle porosity reduction, to reduce dust, for chemical protection, to mask taste, to reduce odor, to minimize gastrointestinal irritation, etc. The isolating effect is proportional to the thickness of the coating. Water soluble cellulose ethers are preferred for this application. HPMC and ethyl cellulose in combination, or Eudragit E100, may be particularly suitable for taste masking applications. Traditional enteric coating materials listed elsewhere can also be applied to form an isolating layer.

Extended release coatings are designed to effect delivery over an extended period of time. The extended release coating is a pH-independent coating formed of, for example, ethyl cellulose, hydroxypropyl cellulose, methylcellulose, hydroxymethyl cellulose, hydroxyethyl cellulose, acrylic esters, or sodium carboxymethyl cellulose. Various extended release dosage forms can be readily designed by one skilled in art to achieve delivery to both the small and large intestines, to only the small intestine, or to only the large intestine, depending upon the choice of coating materials and/or coating thickness.

Enteric coatings are mixtures of pharmaceutically acceptable excipients which are applied to, combined with, mixed with or otherwise added to the carrier or composition. The coating may be applied to a compressed or molded or extruded tablet, a gelatin capsule, and/or pellets, beads, granules or particles of the carrier or composition. The coating may be applied through an aqueous dispersion or after dissolving in appropriate solvent. Additional additives and their levels, and selection of a primary coating material or materials will depend on the following properties: 1. resistance to dissolution and disintegration in the stomach; 2. impermeability to gastric fluids and drug/carrier/enzyme while in the stomach; 3. ability to dissolve or disintegrate rapidly at the target intestine site; 4. physical and chemical stability during storage; 5. non-toxicity; 6. easy application as a coating (substrate friendly); and 7. economical practicality.

Dosage forms of the compositions of the present invention can also be formulated as enteric coated delayed release oral dosage forms, i.e., as an oral dosage form of a pharmaceutical composition as described herein which utilizes an enteric coating to affect release in the lower gastrointestinal tract. The enteric coated dosage form may be a compressed or molded or extruded tablet/mold (coated or uncoated) containing granules, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

Delayed release generally refers to the delivery so that the release can be accomplished at some generally predictable location in the lower intestinal tract more distal to that which would have been accomplished if there had been no delayed release alterations. The preferred method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention to achieve delivery to the lower gastrointestinal tract. Polymers for use in the present invention are anionic carboxylic polymers.

Shellac, also called purified lac, a refined product obtained from the, resinous secretion of an insect. This coating dissolves in media of pH>7.

Colorants, detackifiers, surfactants, antifoaming agents, lubricants, stabilizers such as hydroxy propyl cellulose, acid/base may be added to the coatings besides plasticizers to solubilize or disperse the coating material, and to improve coating performance and the coated product.

In carrying out the method of the present invention, the combination of the invention may be administered to mammalian species, such as dogs, cats, humans, etc. and as such may be incorporated in a conventional systemic dosage form, such as a tablet, capsule, elixir or injectable. The above dosage forms will also include the necessary carrier material, excipient, lubricant, buffer, antibacterial, bulking agent (such as mannitol), anti-oxidants (ascorbic acid of sodium bisulfite) or the like.

The dose administered must be carefully adjusted according to age, weight and condition of the patient, as well as the route of administration, dosage form and regimen and the desired result.

The pharmaceutical compositions of the invention may be administered in the dosage forms in single or divided doses of one to four times daily. It may be advisable to start a patient on a low dose combination and work up gradually to a high dose combination.

Tablets of various sizes can be prepared, e.g., of about 2 to 2000 mg in total weight, containing one or both of the active pharmaceutical ingredients, with the remainder being a physiologically acceptable carrier of other materials according to accepted pharmaceutical practice. These tablets can be scored to provide for fractional doses. Gelatin capsules can be similarly formulated.

Liquid formulations can also be prepared by dissolving or suspending one or the combination of active substances in a conventional liquid vehicle acceptable for pharmaceutical administration so as to provide the desired dosage in one to four teaspoonful.

Dosage forms can be administered to the patient on a regimen of, for example, one, two, three, four, five, six, or other doses per day

In order to more finely regulate the dosage schedule, the active substances may be administered separately in individual dosage units at the same time or carefully coordinated times. Since blood levels are built up and maintained by a regulated schedule of administration, the same result is achieved by the simultaneous presence of the two substances. The respective substances can be individually formulated in separate unit dosage forms in a manner similar to that described above.

In formulating the compositions, the active substances, in the amounts described above, may be compounded according to accepted pharmaceutical practice with a physiologically acceptable vehicle, carrier, excipient, binder, preservative, stabilizer, flavor, etc., in the particular type of unit dosage form.

Illustrative of the adjuvants which may be incorporated in tablets are the following: a binder such as gum tragacanth, acacia, corn starch or gelatin; an excipient such as dicalcium phosphate or cellulose; a disintegrating agent such as corn starch, potato starch, alginic acid or the like; a lubricant such as stearic acid or magnesium stearate; a sweetening agent such as sucrose, aspartame, lactose or saccharin; a flavoring agent such as orange, peppermint, oil of wintergreen or cherry. When the dosage unit form is a capsule, it may contain in addition to materials of the above type a liquid carrier such as a fatty oil. Various other materials may be present as coatings or to otherwise modify the physical form of the dosage unit. For instance, tablets or capsules may be coated with shellac, sugar or both. A syrup of elixir may contain the active compound, water, alcohol or the like as the carrier, glycerol as solubilizer, sucrose as sweetening agent, methyl and propyl parabens as preservatives, a dye and a flavoring such as cherry or orange.

The formulations as described above may be administered for a prolonged period, that is, for as long as the potential for an allergic disease or condition remains or the symptoms continue. In a combination product of antihistamine with wakefulness promoting agent, for example, diphenhydramine has a much shorter pharmacokinetic half life than modafinil, it is beneficial to produce a unique formulation that sustains or slow down the release of diphenhydramine while speeds up the release of modafinil, in order for the combination product to synchronize their two actives' pharmacokinetic profile. Another example of the formulation in the invention is to have a once daily pharmaceutical antihistamine product that sustains the antihistamine (e.g. diphenhydramine) release for as long as 24 hours while the wakefulness promoting agent (e.g.: modafinil) for only 12 hours. Such formulation provides the benefit of 24 hour antihistamine effect but only 12 hr wakefulness effect, thus enables patients sound sleep at night time.

The formulations of the present invention may be formulated as a transdermal delivery system, such as transdermal patches. In certain embodiments of the present invention, a transdermal patch comprises antihistamines and a wakefulness promoting agent in a reservoir or a matrix, and an adhesive which allows the transdermal device to adhere to the skin, allowing the passage of the active agent from the transdermal device through the skin of the mammal.

The AM (or day time or midday) dose formulation of the present invention contains 6-150 mg Diphenhydramine HCl and 50-400 mg modafinil (or 25-275 mg armodafinil), while the PM (or night time) dose contains 6-150 mg diphenhydramine HCL alone.

In another aspect, the AM (or day time or midday) dose formulation of the present invention contains 6-150 mg Diphenhydramine HCl and 25-500 mg Caffeine, while the PM (or night time) dose contains 6-150 mg diphenhydramine HCL alone.

In one aspect, the oral dosage form is a member selected from the group consisting of a capsule, a tablet, a wafer, a chewable tablet, a buccal tablet, a sub lingual tablet, a quick-dissolve tablet, an effervescent tablet, a granule, a gum, a pellet, a bead, a pill, a sachet, a sprinkle, a syrup, a dry syrup, a reconstitutable solid, a suspension, a lozenge, a troche, an oral suspension, a lozenge, an implant, a powder, a triturate, an enterics/controlled release coated tablet, a thin film, and a strip.

In another aspect, a pharmaceutical dosage form comprising an immediate release and a controlled release component, wherein said immediate release component comprises a wakefulness promoting agent, or a pharmaceutically acceptable salt or derivative thereof; wherein said controlled release component comprises an antihistamine, or a pharmaceutically acceptable salt or derivative thereof; wherein said immediate release component exhibits an in vitro dissolution profile comprising at least about 10% wakefulness promoting agent release after 15 minutes and at least about 60% wakefulness promoting agent release after 1 hour; and wherein said controlled release component exhibits an in vitro dissolution profile comprising from about 10% to about 60% antihistamine release after 1 hour, from about 20% to about 80% antihistamine release after 2 hours, and at least about 30% antihistamine release after 6 hours.

### Packaging/Treatment Kits

The present invention relates to a kit for conveniently and effectively carrying out the methods in accordance with the present invention. Such kits may be suited for the delivery of solid oral forms such as tablets or capsules. Such a kit may include a number of unit dosages. Such kits can include a means for containing the dosages oriented in the order of their intended use. An example of a means for containing the dosages in the order of their intended uses is a card. An example of such a kit is a “blister pack”. Blister packs are well known in the packaging industry and are widely used for packaging pharmaceutical unit dosage forms. If desired, the blister can be in the form of a childproof blister, i.e. a blister that is difficult for a child to open, yet can be readily opened by an adult. If desired, a memory aid can be provided, for example in the form of numbers, letters, or other markings or with a calendar feature and/or calendar insert, designating the days and the sections of a day in the treatment schedule in which the dosages can be administered, such as an AM dose is packaged with a “mid day” and a PM dose.; or an AM dose is packaged with a PM dose. Alternatively, placebo dosages, or vitamin or dietary supplements, either in a form similar to or distinct from the pharmaceutical active dosages, can be included.

The invention provides compositions, including preparations, formulations and/or kits, comprising combinations of ingredients, as described above (including the multi-ingredient combinations of drugs of the invention), that are serviceable as therapies for treating, preventing or improving conditions, states and disease symptoms involving allergies and/or allergic conditions related thereto. In one aspect, each member of the combination of ingredients is manufactured in a separate package, kit or container; or, all or a subset of the combinations of ingredients are manufactured in a separate package or container. In alternative aspects, the package, kit or container comprises a blister package, a clamshell, a tray, a shrink wrap and the like.

In one aspect, the package, kit or container comprises a “blister package” (also called a blister pack, or bubble pack). In one aspect, the blister package consists two or more separate compartments: Am dosage of this invention, and PM dosage of this invention, or mid-day dosage of this invention. This blister package is made up of two separate material elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, the invention provides for blister packs, clamshells or trays comprising a composition (e.g., a (the multi-ingredient combination of drugs of the invention) combination of active ingredients) of the invention. Blister packs, clamshells or trays can be designed to be non-reclosable, so consumers can tell if a package has already opened. They are used to package for sale goods where product tampering is a consideration, such as the pharmaceuticals of the invention. In one aspect, a blister pack of the invention comprises a moulded PVC base, with raised areas (the “blisters”) to contain the tablets, pills, etc. comprising the combinations of the invention, covered by a foil laminate. Tablets, pills, etc. are removed from the pack either by peeling the foil back or by pushing the blister to force the tablet to break the foil. In one aspect, a specialized form of a blister pack is a strip pack.

In one aspect, a blister pack also comprise a method of packaging where the compositions comprising combinations of ingredients of the invention are contained in-between a card and a clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly coloured and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, e.g., for items such as pills, tablets, geltabs, etc. of the invention are used, and they can comprise of two vacuum-formed PVC sheets meshed together at the edges, with the informative card inside.

In one aspect, blister packaging comprises at least two components (e.g., is a multi-ingredient combination of drugs of the invention): a thermoformed “blister” which houses the product (e.g., a pharmaceutical combination of the invention), and then a “blister card” that is a printed card with an adhesive coating on the front surface. During the assembly process, the blister component, which is most commonly made out of PVC, is attached to the blister card using a blister machine. This machine introduces heat to the flange area of the blister which activates the glue on the card in that specific area and ultimately secures the PVG blister to the printed blister card. The thermoformed PVG blister and the printed blister card can be as small or large. Conventional blister packs can also be sealed (e.g., using an AERGO 8 DUO®, SCA Consumer Packaging, Inc., DeKalb, Ill.) using regular heat seal tooling. This alternative aspect, using heat seal tooling, can seal common types of thermoformed packaging.

For example, in one aspect, the invention provides a blister package, a clamshell, a tray or a shrink wrap comprising at least one an antihistamine, or pharmaceutically acceptable salt or derivative thereof and at least one wakefulness promoting agent, or pharmaceutically acceptable salt or derivative thereof. In one aspect, the therapeutic combination in the blister package, clamshell, tray or shrink wrap comprises at least one an antihistamine, or pharmaceutically acceptable salt or derivative thereof and at least one wakefulness promoting agent, or pharmaceutically acceptable salt or derivative thereof. In one aspect, the therapeutic combination in the blister package, clamshell, tray or shrink wrap comprises an antihistamine, or pharmaceutically acceptable salt or derivative thereof or a combination thereof, and at least one wakefulness promoting agent, or pharmaceutically acceptable salt or derivative thereof.

As discussed herein, the products of manufacture of the invention can comprise the packaging of the therapeutic drug combinations of the invention, alone or in combination, as “blister packages” or as a plurality of packettes, including as lidded blister packages, lidded blister or blister card or packets, or a shrink wrap.

In one aspect, laminated aluminum foil blister packs are used, e.g., for the preparation of drugs designed to dissolve immediately in the mouth of a patient. This exemplary process comprises having the drug combinations of the invention prepared as an aqueous solution(s) which are dispensed (e.g., by measured dose) into an aluminum (e.g., alufoil) laminated tray portion of a blister pack. This tray is then freeze-dried to form tablets which take the shape of the blister pockets. The alufoil laminate of both the tray and lid fully protects any highly hygroscopic and/or sensitive individual doses. In one aspect, the pack incorporates a child-proof peel open security laminate. In one aspect, the system give tablets an identification mark by embossing a design into the alufoil pocket that is taken up by the tablets when they change from aqueous to solid state. In one aspect, individual ‘push-through’ blister packs/packettes are used, e.g., using hard temper aluminum (e.g., alufoil) lidding material. In one aspect, hermetically-sealed high barrier aluminum (e.g., alufoil) laminates are used. In one aspect, any of the invention's products of manufacture, including kits or blister packs, use foil laminations and strip packs, stick packs, sachets and pouches, peelable and non-peelable laminations combining foil, paper, and film for high barrier packaging.

In one aspect, any of the invention's products of manufacture, including kits or blister packs, include memory aids to help remind patients when and how to take the drug.

This safeguards the drug's efficacy by protecting each pill until it's taken; gives the product or kit portability, makes it easy to take a dose anytime or anywhere.

The treatment kits can be constructed in a variety of forms familiar to one of ordinary skill in the art. The kits comprise at least one unit dosage of a pharmaceutical active for administration according to a daily regimen and a means for containing the unit dosages. The treatment kits can, for example, be constructed for once daily, twice daily, thrice daily, or four times daily administration, or other dosage regimens. The kits comprise a means for the daily administration of a pharmaceutical active. In one embodiment the kits include from about one to about four unit dosages.

In another embodiment, the kits include about one-four unit dosages.

In another embodiment, the kit includes two containers: AM container containing multiple AM dosage units; PM container containing multiple PM dosage units. These two containers maybe physically connected or not connected.

In another embodiment, the kit includes three containers: AM container containing multiple AM dosage units; Mid-Day container containing multiple midday dosage units; PM container containing multiple PM dosage units. These three containers maybe physically connected or not connected.

In one embodiment, the means for containing the unit dosages is a card, including, for example, a card that is capable of being folded. This card will be referred to herein as a main card, or alternatively a principal card or a first card, to distinguish it from additional optional cards, circulars, or other such materials which can be associated with the kit. This main card can be folded with a simple crease, or alternatively, with a double crease, so as to exhibit a spine, similar to the spine of a closed book. The main card can comprise a printable surface, i.e. a surface upon which the product name, appropriate administration instructions, product information, drawings, logos, memory aids, calendar features, etc. can be printed. The main card can comprise a means for containing said unit dosage or different dosages designated for different time of the day, and a memory aid for administering said unit dosage or dosages. The main card, especially if it is prepared from two or more laminated paperboard surfaces, can comprise a slit or pocket, for example in one of the inner paperboard surfaces of the folded card. The slit or pocket can be used to contain a removable secondary card, i.e. a second card or insert card, which is not permanently attached or affixed to the main card.

The memory aid can include a listing of the days of the week, i.e. Sunday, Monday, Tuesday, Wednesday, Thursday, Friday, and Saturday, with appropriate spaces for the patient to select and indicate on the card the preferred day of the week on which to administer the therapy. The memory aid can include a listing of the time of day with appropriate spaces for the patient to select and indicate on the card the preferred time of day (i.e.: AM, PM, midday) at which to administer the therapy. The memory aid can also include removable stickers having an appropriate pressure sensitive adhesive to facilitate easy removal and refastening to a desired surface such as a calendar or dayminder. The removable stickers can be located on the main card, or can be located on the secondary card which is constructed so that it can be readily inserted into and removed from the optional slit in the main card. Additionally, the optional slit can contain additional patient information and other circulars.

Other means for containing said unit dosages can include bottles and vials, wherein the bottle or vial comprises a memory aid, such as a printed label for administering said unit dosage or dosages. The label can also contain removable reminder stickers for placement on a calendar or dayminder to further help the patient to remember when to take a dosage or when a dosage has been taken.

### Personalized Dosages

The dosage regimen utilizing the compounds of the present invention may be selected, for example, in accordance with a variety of factors, including, but not limited to, type, species, age, weight, sex and medical condition of the patient or subject; the severity of the condition to be treated; the route of administration; the renal and hepatic function of the patient or subject; and the particular compound or salt thereof employed. An ordinarily skilled physician, veterinarian or clinician can readily determine and prescribe the effective amount of the compositions required to treat or reduce the risk of contracting the condition.

In another embodiment, the dosage form may be administered as a personalized medicine based on the body weight of an individual, patient or subject. Individuals, patients or subjects may be, for example, categorized according to their gender and body weight. For example, they may be categorized in the following 4 Categories:
Category A: Less than 100 lb
Category B: 95-170 lb
Category C: 150-300 Lb
Category D: over 270 lb
Administration of an AM dosage to Category A patients may be, for example, an AM dosage comprising in the range of about 6-50 mg diphenhydramine and a wakefulness promoting agent selected from the group consisting of about 50-150 mg modafinil, about 25-100 mg armodafinil, about 50-150 mg Caffeine per dosage unit.
Administration of an AM dosage to Category B patients may be, for example, an AM dosage comprising in the range of about 25-75 mg diphenhydramine and a wakefulness promoting agent selected from the group consisting of about 100-200 mg modafinil, 75-150 mg armodafinil, and 130-240 mg Caffeine per dosage unit.
Administration of an AM dosage to Category C patients may be, for example, an AM dosage comprising in the range of about 50-100 mg diphenhydramine and a wakefulness promoting agent selected from the group consisting of about 150-250 mg modafinil, 125-200 mg armodafinil, 220-340 mg Caffeine per dosage unit.
Administration of an AM dosage to Category D patients may be, for example, an AM dosage comprising in the range of about 75-125 mg diphenhydramine and a wakefulness promoting agent selected from the group consisting of about 200-350 mg modafinil, 150-225 mg armodafinil, 310-425 mg Caffeine per dosage unit.
Administration of PM dosage to Category A patients may be for example in the range of about 6-50 mg diphenhydramine per dosage unit.
Administration of PM dosage to Category B patients may be for example in the range of about 25-75 mg diphenhydramine per dosage unit.
Administration of PM dosage to Category C patients may be for example in the range of about 50-100 mg diphenhydramine per dosage unit.
Administration of PM dosage to Category D patients may be for example in the range of about 75-125 mg diphenhydramine per dosage unit.

The dosage regimen utilizing the compositions of the present invention may be dispensed daily (usually at 9:00 a.m.), bid (twice a day), tid (three times per day), qid (fourtimes per day), q6° (every six hours), or q8° (every eight hours). The exact times at which the medication is dispensed may vary with the lifestyle of the patient. Someone who is an early riser and on a qid schedule may choose a 9:00 a.m., 3:00 p.m., 9:00 p.m., 3:00 a.m. medication dispensing schedule instead of a more usual 6:00 a.m., 12:00 noon, 6:00 p.m., 12:00 a.m. schedule.

The present invention relates to a method and system for packaging and administering an individualized regime of therapeutic dosages comprising marking a plurality of separate dosages with individual dosage designations corresponding to the time and/or day and/or date of administration, wherein the dosage can be selectively removed from the dosage package from the corresponding position at the appropriate time and day or date for administering the appropriate therapeutic dosage.

The set indicia on the package may comprise an indicator such as Monday or MON, Tuesday or TUES, Wednesday or WED, Thursday or THUR, Friday or FRI, Saturday or SAT, Sunday or SUN, a numeric indicator such as 1st, 2nd, 3rd, 4th, 5th, 6th, 7th, discrete differentiating color indicators, discrete differentiating Braille indicators or other such differentiating indicators.

The indicia on the package may comprise an indicator such as AM, NN (mid-day), PM and BT (bedtime), discrete differentiating color indicators such as green, black, blue and red respectively, discrete differentiating Braille indicators or other such differentiating indicators.

The package may have a plurality of positions comprising packet compartments formed in columns and rows each including the code comprising a set indicia and a subset indicia formed thereon.

The term “AM dose” refers to, for example, dosages given during daytime, first dose after getting up, and/or any dose given during daytime. The term “Mid day dose” refers to, for example, dosages given any time after the first dose after getting up, but before the last dose before bed. The term “PM dose” refers to, for example, dosages given last dose before bedtime. The term “daytime dose” refers to, for example, dosages given at any time other than the bedtime dose. The term “bedtime dose” refers to, for example, the last dosage given before bedtime.

As used in this specification and in the appended claims, the term “pharmaceutical” is meant to be understood broadly as any medicinal structure or edible casing configured to house a substance related to a medicinal treatment. The medicinal structure can include an active ingredient for an approved medical treatment, a medical treatment being evaluated, or a placebo ingredient used during clinical trials to compare against the medical treatment being evaluated (i.e., a placebo capsule).

The invention will be illustrated in more detail with reference to the following Examples, but it should be understood that the present invention is not deemed to be limited thereto.

### EXAMPLES

The following examples describe exemplary treatment protocols of the invention, using exemplary therapeutic combination compositions of the invention.

### Example 1

Extended-release formulation of diphenhydramine and modafinil, diphenhydramine and armodafinil, and diphenhydramine and caffeine. The ingredients as set forth in Table 1, excluding the lubricant are mixed in a high shear granulator. Water is added and the mixture wet granulated. The granulation is screened, dried, and milled. The granulation is added into a low shear blender, the lubricant is added, and blended. The final blend is compressed on a tablet press to form extended-release dosage forms.

TABLE 1

| Example 1. Extended- | | | |
|---|---|---|---|
| release formulation of | | | |
| diphenhydramine & | | | |
| modafinil or | | | Formulation |
| armodafinil or Caffeine | Formulation A | Formulation B | C |
| Ingredient | Weight (mg) | Weight (mg) | Weight (mg) |
| diphenhydramine HCl | 75 | 75 | 75 |
| Modafinil | 200 | | |
| Armodafinil | | 150 | |
| Caffeine | | | 240 |
| Carbopol 971P NF | 460 | 460 | 460 |
| polymer | | | |
| Lactose monohydrate | 40 | 40 | 40 |
| Talc | 5 | 5 | 5 |
| Magnesium Stearate | 5 | 5 | 5 |
| (lubricant) | | | |
| Total | 785 | 735 | 825 |

### Example 2

Extended-release formulation of diphenhydramine & modafinil or armodafinil or Caffeine. The ingredients as set forth in Table 2 excluding the lubricant and glidant are mixed in a low shear blender for 20 minutes. The lubricant and glidant are added and blended for 5 minutes. The formulation is directly compressed on a tablet press.

TABLE 2

| | | | Formu- |
|---|---|---|---|
| | Formulation | Formulation | lation C |
| | A | B | Weight |
| Ingredient | Weight (mg) | Weight (mg) | (mg) |
| Diphenhydramine | 50 | 50 | 50 |
| Modafinil | 150 | | |
| Armodafinil | | 100 | |
| Caffeine | | | 150 |
| Hydroxypropylmethylcellulose | 460 | 460 | 460 |
| Lactose monohydrate | 40 | 40 | 40 |
| Colloidal Silicon Dioxide | 5 | 5 | 5 |
| (Glidant) | | | |
| Magnesium Stearate | 5 | 5 | 5 |
| (Lubricant) | | | |
| Total | 710 | 660 | 710 |

### Example 3

Extended-release formulation of diphenhydramine & modafinil or armodafinil or Caffeine. The ingredients as et forth in Table 3 excluding the lubricant are mixed in a high shear granulator. Water and ethyl alcohol are added as a granulating solution and the mixture wet granulated. The granulation is screened, dried, and milled. The granulation is added into a low shear blender, the glidant and lubricant are added, and blended. The final blend is compressed on a tablet press.

TABLE 3

| | | | Formu- |
|---|---|---|---|
| | Formulation | Formulation | lation C |
| | A | B | Weight |
| Ingredient | Weight (mg) | Weight (mg) | (mg) |
| Diphenhydramine | 100 | 100 | 100 |
| Modafinil | 250 | | |
| Armodafinil | | 200 | |
| Caffeine | | | 340 |
| Hydroxypropylmethylcellulose | 200 | 200 | 200 |
| Hydroxyethylcellulose | 260 | 260 | 260 |
| Lactose monohydrate | 40 | 40 | 40 |
| Colloidal Silicon Dioxide | 5 | 5 | 5 |
| (Glidant) | | | |
| Magnesium Stearate | 5 | 5 | 5 |
| (Lubricant) | | | |
| Total | 860 | 810 | 950 |

### Example 4

Extended-release wax formulation. The ingredients as set forth in Table 4 excluding the lubricant and glidant are mixed in a high shear granulator. Water and ethyl alcohol are added and the mixture wet granulated. The granulation is screened, dried, and milled. The granulation is added into a low shear blender, the glidant and lubricant are added, and blended. The final blend is compressed on a tablet press.

TABLE 4

| | Formulation A | Formulation B | Formulation C |
|---|---|---|---|
| Ingredient | Weight (mg) | Weight (mg) | Weight (mg) |
| Diphenhydramine | 50 | 50 | 50 |
| Modafinil | 175 | | |
| Armodafinil | | 125 | |
| Caffeine | | | 150 |
| Carnauba Wax | 460 | 460 | 460 |
| Microcrystalline | 40 | 40 | 40 |
| Cellulose | | | |
| Colloidal Silicon | 5 | 5 | 5 |
| Dioxide (Glidant) | | | |
| Magnesium Stearate | 5 | 5 | 5 |
| (Lubricant) | | | |
| Total | 735 | 685 | 710 |

### Example 5

A study was conducted on one female patient, body wt at 120 lb, 41 year of age. The patient is a long time allergy sufferer. During the study, the patient was given the following doses:

Treatment A
9 am on a study day: 50 mg diphenhydramine together with 100 mg Provigil(® (modafinil); 3 pm on the study day: 50 mg diphenhydramine alone;
10 pm on the study day: 50 mg diphenhydramine alone.
During this treatment, the subject had no allergic symptoms, had excellent night time rest, and had negligible drowsiness during waking hours.

Treatment B
On another study day, the same patient was given the following dose regimen:
9 am on the study day: 50 mg diphenhydramine together with 100 mg Provigil® (modafinil);
10 pm on the study day: 50 mg diphenhydramine alone. During this treatment, the patient had minimal allergic symptoms, had an excellent night time rest, and had negligible drowsiness during waking hours.

Treatment C
On another study day, the same patient was given the following dose regimen:
9 am on the study day: 50 mg diphenhydramine together with 150 mg Caffeine;
3 pm on the study day: 50 mg diphenhydramine together with 150 mg Caffeine;
10 pm on the study day: 50 mg diphenhydramine alone

During this treatment, the patient had no allergic symptoms, had a good night time rest, and had negligible drowsiness during waking hours.

Treatment D
On another study day, the same patient was given the following dose regimen:
9 am on the study day: 50 mg diphenhydramine together with 100 mg Provigil® (modafinil);
10 pm on the study day: 50 mg diphenhydramine together with 100 mg Provigil® (modafinil)

During this treatment, the subject had minimal allergic symptoms, was not able to sleep at night time, and had negligible drowsiness during waking hours.

### Example 6

This example demonstrates a personalized pharmaceutical packaging system comprising: a therapeutic dosage to be administered in the AM, which comprises a tablet containing the ingredients from Example 2, formulation C: 50 mg Diphenhydramine HCL/150 mg Caffeine; a therapeutic dosage to be administered in the midday, which comprises a tablet containing 50 mg diphenhydramine HCl; and a therapeutic dosage to be administered in the PM, which comprises a tablet containing 50 mg diphenhydramine HCl.

### Example 7

This example demonstrates a personalized pharmaceutical packaging system comprising: a therapeutic dosage to be administered in the AM, which comprises a tablet formulation contains the ingredients from Example 2, formulation C: 50 mg Diphenhydramine HCL/150 mg Caffeine; a therapeutic dosage to be administered in the midday, which comprises a tablet formulation contains the ingredients from Example 2, formulation C: 50 mg Diphenhydramine HCL/150 mg Caffeine; and a therapeutic dosage to be administered in the PM, which comprises a tablet containing 50 mg diphenhydramine HCl.

### Example 8

This example demonstrates a personalized pharmaceutical packaging system comprising: a therapeutic dosage to be administered in the AM, which comprises a tablet formulation with the ingredients from Example 2, formulation A: 50 mg Diphenhydramine HCL/150 mg Modafinil; a therapeutic dosage to be administered in the midday, which comprises a tablet formulation contains the ingredients from Example 1, formulation C: 50 mg Diphenhydramine HCL/150 mg Caffeine; and a therapeutic dosage to be administered in the PM, which comprises a tablet containing 50 mg diphenhydramine HCl.

### Example 9

This example demonstrates a personalized pharmaceutical packaging system comprising: a therapeutic dosage to be administered in the AM, which comprises a tablet formulation, which contains the ingredients from Example 1, formulation A: 75 mg Diphenhydramine HCL/200 mg Modafinil; a therapeutic dosage to be administered in the PM, which comprises a tablet containing 50 mg diphenhydramine HCl.

### Example 10

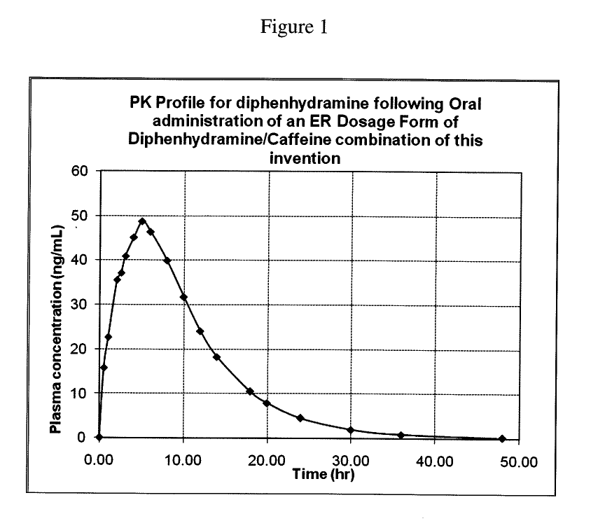

Referring to FIG. 1, this example demonstrates the pharmacokinetic profile of diphenhydramine from the extended release formulation of the AM formulation listed under Example 2 formulation C: 50 mg Diphenhydramine HCL/150 mg Caffeine.

### Example 11

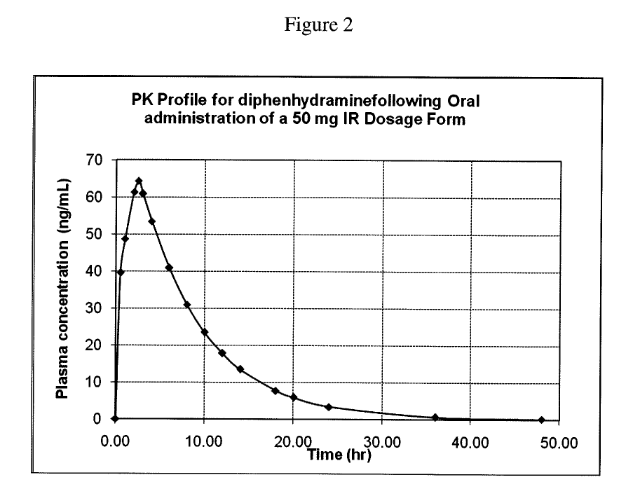

Referring to FIG. 2, this example demonstrates the pharmacokinetic profile of diphenhydramine from an immediate release diphenhydramine pharmacokinetics.

While the invention has been described in detail and with reference to specific examples thereof, it will be apparent to one skilled in the art that various changes and modifications can be made therein without departing from the spirit and scope thereof.

1. A personalized pharmaceutical packaging system for administering a plurality of pharmaceutical active ingredients to an individual in need thereof according to a personalized daily regimen, said personalized pharmaceutical packaging system comprising:
a therapeutic dosage to be administered in the AM, said therapeutic dosage comprising first active ingredient of at least one antihistamine, or a pharmaceutically acceptable salt or derivative thereof, and second active ingredient of at least one wakefulness promoting agent, or a pharmaceutically acceptable salt or derivative thereof, wherein the first and second active ingredients are in multiple separated dosage units, or in a single dosage unit comprising a combination of active ingredients;
optionally, a therapeutic dosage to be administered in the midday, comprising a first active ingredient of at least one antihistamine, or a pharmaceutically acceptable salt or derivative thereof, and an optional second active ingredient of at least one wakefulness promoting agent, or a pharmaceutically acceptable salt or derivative thereof, wherein the first and second active ingredients are in multiple separated dosage units, or in a single dosage unit comprising a combination of active ingredients;
a therapeutic dosage to be administered in the PM or bedtime, comprising at least one unit dosage comprising at least one antihistamine, or a pharmaceutically acceptable salt or derivative thereof;
a means for containing said unit dosage or dosages, wherein said means for containing said unit dosage or dosages further comprises: (i) at least one printable surface; and/or (ii) a memory aid for administering said unit dosage or dosages; and/or (iii) directions for administering the unit dosage or dosages; and
optionally wherein the personalized dosage regimen is customized for the needs of the individual based on criteria relevant to the individuals personal information.

2. The personalized pharmaceutical packaging system of claim 1, wherein the personal information comprises any one or combination of the following:
the ethnicity of the individual;
the sex of the individual;
the weight of the individual;
the Body Mass Index of the individual:
age of the individual;
the incidence of a condition of potential interest for the personalized therapeutic regimen in the individual's family;
the environmental conditions of the individual; and
combinations thereof.

3. The personalized pharmaceutical packaging system of claim 1, wherein the personalized dosage regimen is based on the body weight of the individual, and is selected from the group consisting of:
an AM dosage for an individual less than 100 lb, comprising a range of about 6-50 mg diphenhydramine and a wakefulness promoting agent selected from the group consisting of adrafinil, about 50-150 mg modafinil, about 25-100 mg armodafinil, and about 50-150 mg Caffeine per dosage unit;
an AM dosage for an individual of 95-170 lb, comprising a range of about 25-75 mg diphenhydramine and a wakefulness promoting agent selected from the group consisting of adrafinil, about 100-200 mg modafinil, about 75-150 mg armodafinil, and about 130-240 mg Caffeine per dosage unit;
an AM dosage for an individual of 150-300 lb, comprising a range of about 50-100 mg diphenhydramine and a wakefulness promoting agent selected from the group consisting of adrafinil, about 150-250 mg modafinil, 125-200 mg armodafinil, and about 220-340 mg Caffeine per dosage unit;
an AM dosage for an individual over 270 lb, comprising a range of about 75-125 mg diphenhydramine and a wakefulness promoting agent selected from the group consisting of adrafinil, about 200-350 mg modafinil, about 150-225 mg armodafinil, and about 310-425 mg Caffeine per dosage unit;
a PM dosage for an individual less than 100 lb, comprising a range of about 6-50 mg diphenhydramine per dosage unit;
a PM dosage for an individual of 95-170 lb, comprising a range of about 25-75 mg diphenhydramine per dosage unit;
a PM dosage for an individual of 150-300 lb, comprising a range of about 50-100 mg diphenhydramine per dosage unit; and
a PM dosage for an individual over 270 lb, comprising a range of about 75-125 mg diphenhydramine per dosage unit.

4. The personalized pharmaceutical packaging system of claim 1, wherein the means for containing said AM, PM or MIDDAY therapeutic dosage or dosages is selected from the group consisting of:
the first and second unit dosages are packaged together in a single package or packette;
the first and second unit dosages are packaged separately in a plurality of packages or packettes; a blister packet; a lidded blister; or blister card or packets;
a shrink wrap, and with both drugs released upon opening of the single package or packette;
a plurality of packages or packettes; blister packet; lidded blister or blister card or packets; or shrink wrap;
a blister pack;
a container; and
a device,
and further wherein the AM, PM and optional MIDDAY dosages are separated from each other within the personalized pharmaceutical packaging system.

5. The personalized pharmaceutical packaging system of claim 1 wherein said system is selected from the group consisting of:
a connected bottle pack comprising an AM bottle, a PM bottle, and an optional MIDDAY bottle; and
a blister package comprising: a) a rupturable substrate b) a layer forming one or more blisters over the rupturable substrate, wherein each of the one or more blisters contain one or more unit dosage forms.

6. A method of making a personalized pharmaceutical packaging system for administering a plurality of pharmaceutical active ingredients to an individual in need thereof according to a personalized daily regimen, said personalized pharmaceutical packaging system comprising:
a therapeutic dosage to be administered in the AM, said therapeutic dosage comprising first active ingredient of at least one antihistamine, or a pharmaceutically acceptable salt or derivative thereof, and second active ingredient of at least one wakefulness promoting agent, or a pharmaceutically acceptable salt or derivative thereof, wherein the first and second active ingredients are in multiple separated dosage units, or in a single dosage unit comprising a combination of active ingredients;
optionally, a therapeutic dosage to be administered in the midday, comprising a first active ingredient of at least one antihistamine, or a pharmaceutically acceptable salt or derivative thereof, and an optional second active ingredient of at least one wakefulness promoting agent, or a pharmaceutically acceptable salt or derivative thereof, wherein the first and second active ingredients are in multiple separated dosage units, or in a single dosage unit comprising a combination of active ingredients;
a therapeutic dosage to be administered in the PM or bedtime, comprising at least one unit dosage comprising at least one antihistamine, or a pharmaceutically acceptable salt or derivative thereof;
a means for containing said unit dosage or dosages, wherein said means for containing said unit dosage or dosages further comprises: (i) at least one printable surface; and/or (ii) a memory aid for administering said unit dosage or dosages; and/or (iii) directions for administering the unit dosage or dosages; and
optionally wherein the personalized dosage regimen is customized for the needs of the individual based on criteria relevant to the individuals personal information.

7. The method of making a personalized pharmaceutical packaging system of claim 6, wherein the personal information comprises any one or combination of the following:
the ethnicity of the individual;
the sex of the individual;
the weight of the individual;
the Body Mass Index of the individual:
age of the individual;
the incidence of a condition of potential interest for the personalized therapeutic regimen in the individual's family;
the environmental conditions of the individual; and
combinations thereof.

8. The method of making a personalized pharmaceutical packaging system of claim 6, wherein the personalized dosage regimen is based on the body weight of the individual, and is selected from the group consisting of:
an AM dosage for an individual less than 100 lb, comprising a range of about 6-50 mg diphenhydramine and a wakefulness promoting agent selected from the group consisting of adrafinil, about 50-150 mg modafinil, about 25-100 mg armodafinil, and about 50-150 mg Caffeine per dosage unit;
an AM dosage for an individual of 95-170 lb, comprising a range of about 25-75 mg diphenhydramine and a wakefulness promoting agent selected from the group consisting of adrafinil, about 100-200 mg modafinil, about 75-150 mg armodafinil, and about 130-240 mg Caffeine per dosage unit;
an AM dosage for an individual of 150-300 lb, comprising a range of about 50-100 mg diphenhydramine and a wakefulness promoting agent selected from the group consisting of adrafinil, about 150-250 mg modafinil, 125-200 mg armodafinil, and about 220-340 mg Caffeine per dosage unit;
an AM dosage for an individual over 270 lb, comprising a range of about 75-125 mg diphenhydramine and a wakefulness promoting agent selected from the group consisting of adrafinil, about 200-350 mg modafinil, about 150-225 mg armodafinil, and about 310-425 mg Caffeine per dosage unit;
a PM dosage for an individual less than 100 lb, comprising a range of about 6-50 mg diphenhydramine per dosage unit;
a PM dosage for an individual of 95-170 lb, comprising a range of about 25-75 mg diphenhydramine per dosage unit;
a PM dosage for an individual of 150-300 lb, comprising a range of about 50-100 mg diphenhydramine per dosage unit; and
a PM dosage for an individual over 270 lb, comprising a range of about 75-125 mg diphenhydramine per dosage unit.

9. The method of making a personalized pharmaceutical packaging system of claim 6, wherein the means for containing said AM, PM or MIDDAY therapeutic dosage or dosages is selected from the group consisting of:
the first and second unit dosages are packaged together in a single package or packette;
the first and second unit dosages are packaged separately in a plurality of packages or packettes; a blister packet; a lidded blister; or blister card or packets;
a shrink wrap, and with both drugs released upon opening of the single package or packette;
a plurality of packages or packettes; blister packet; lidded blister or blister card or packets; or shrink wrap;
a blister pack;
a container; and
a device,
and further wherein the AM, PM and optional MIDDAY dosages are separated from each other within the personalized pharmaceutical packaging system.

10. The method of making a personalized pharmaceutical packaging system of claim 6 wherein said system is selected from the group consisting of:
a connected bottle pack comprising an AM bottle, a PM bottle, and an optional MIDDAY bottle; and
a blister package comprising: a) a rupturable substrate b) a layer forming one or more blisters over the rupturable substrate, wherein each of the one or more blisters contain one or more unit dosage forms.

11. A method of treating a disease or condition in mammal by using a personalized pharmaceutical packaging system for administering a plurality of pharmaceutical active ingredients to an individual in need thereof according to a personalized daily regimen, said personalized pharmaceutical packaging system comprising:
a therapeutic dosage to be administered in the AM, said therapeutic dosage comprising first active ingredient of at least one antihistamine, or a pharmaceutically acceptable salt or derivative thereof, and second active ingredient of at least one wakefulness promoting agent, or a pharmaceutically acceptable salt or derivative thereof, wherein the first and second active ingredients are in multiple separated dosage units; or in a single dosage unit of combination active ingredients;
optionally, a therapeutic dosage to be administered in the midday, comprising a first active ingredient of at least one antihistamine, or a pharmaceutically acceptable salt or derivative thereof, and an optional second active ingredient of at least one wakefulness promoting agent, or a pharmaceutically acceptable salt or derivative thereof, wherein the first and second active ingredients are in multiple separated dosage units, or in a single dosage unit comprising a combination of active ingredients;
a therapeutic dosage to be administered in the PM or bedtime, comprising at least one unit dosage comprising at least one antihistamine, or a pharmaceutically acceptable salt or derivative thereof;
a means for containing said unit dosage or dosages, wherein said means for containing said unit dosage or dosages further comprises: (i) at least one printable surface; and/or (ii) a memory aid for administering said unit dosage or dosages; and/or (iii) directions for administering the unit dosage or dosages; and
optionally wherein the personalized dosage regimen is customized for the needs of the individual based on criteria relevant to the individuals personal information.

12. The method of treating a disease or condition in mammal according to claim 11 by using a personalized pharmaceutical packaging system, wherein the personal information comprises any one or combination of the following:
the ethnicity of the individual;
the sex of the individual;
the weight of the individual;
the Body Mass Index of the individual:
age of the individual;
the incidence of a condition of potential interest for the personalized therapeutic regimen in the individual's family;
the environmental conditions of the individual; and
combinations thereof.

13. The method of treating a disease or condition in mammal according to claim 11 by using a personalized pharmaceutical packaging system, wherein the personalized dosage regimen is based on the body weight of the individual, and is selected from the group consisting of:
an AM dosage for an individual less than 100 lb, comprising a range of about 6-50 mg diphenhydramine and a wakefulness promoting agent selected from the group consisting of adrafinil, about 50-150 mg modafinil, about 25-100 mg armodafinil, and about 50-150 mg Caffeine per dosage unit;
an AM dosage for an individual of 95-170 lb, comprising a range of about 25-75 mg diphenhydramine and a wakefulness promoting agent selected from the group consisting of adrafinil, about 100-200 mg modafinil, about 75-150 mg armodafinil, and about 130-240 mg Caffeine per dosage unit;
an AM dosage for an individual of 150-300 lb, comprising a range of about 50-100 mg diphenhydramine and a wakefulness promoting agent selected from the group consisting of adrafinil, about 150-250 mg modafinil, 125-200 mg armodafinil, and about 220-340 mg Caffeine per dosage unit;
an AM dosage for an individual over 270 lb, comprising a range of about 75-125 mg diphenhydramine and a wakefulness promoting agent selected from the group consisting of adrafinil, about 200-350 mg modafinil, about 150-225 mg armodafinil, and about 310-425 mg Caffeine per dosage unit;
a PM dosage for an individual less than 100 lb, comprising a range of about 6-50 mg diphenhydramine per dosage unit;
a PM dosage for an individual of 95-170 lb, comprising a range of about 25-75 mg diphenhydramine per dosage unit;
a PM dosage for an individual of 150-300 lb, comprising a range of about 50-100 mg diphenhydramine per dosage unit; and
a PM dosage for an individual over 270 lb, comprising a range of about 75-125 mg diphenhydramine per dosage unit.

14. The method of treating a disease or condition in mammal according to claim 11 by using a personalized pharmaceutical packaging system, wherein the means for containing said AM, PM or MIDDAY therapeutic dosage or dosages is selected from the group consisting of:
the first and second unit dosages are packaged together in a single package or packette;
the first and second unit dosages are packaged separately in a plurality of packages or packettes; a blister packet; a lidded blister; or blister card or packets;
a shrink wrap, and with both drugs released upon opening of the single package or packette;
a plurality of packages or packettes; blister packet; lidded blister or blister card or packets; or shrink wrap;
a blister pack;
a container; and
a device,
and further wherein the AM, PM and optional MIDDAY dosages are separated from each other within the personalized pharmaceutical packaging system.

15. The method of treating a disease or condition in mammal according to claim 11 by using a personalized pharmaceutical packaging system, wherein said system is selected from the group consisting of:
a connected bottle pack comprising an AM bottle, a PM bottle, and an optional MIDDAY bottle; and
a blister package comprising: a) a rupturable substrate b) a layer forming one or more blisters over the rupturable substrate, wherein each of the one or more blisters contain one or more unit dosage forms.

16. A pharmaceutical unit dosage form according to claim 3 wherein the dosage is selected from the group consisting of:
an AM dosage for an individual less than 100 lb, comprising a range of about 6-50 mg diphenhydramine, and a wakefulness promoting agent selected from the group consisting of adrafinil, about 50-150 mg modafinil, about 25-100 mg armodafinil, and about 50-150 mg Caffeine per dosage unit;
an AM dosage for an individual of 95-170 lb, comprising a range of about 25-75 mg diphenhydramine a wakefulness promoting agent selected from the group consisting of adrafinil, about 100-200 mg modafinil, about 75-150 mg armodafinil, and about 130-240 mg Caffeine per dosage unit;
an AM dosage for an individual of 150-300 lb, comprising a range of about 50-100 mg diphenhydramine and a wakefulness promoting agent selected from the group consisting of adrafinil, about 150-250 mg modafinil, about 125-200 mg armodafinil, and about 220-340 mg Caffeine per dosage unit;
an AM dosage for an individual over 270 lb, comprising a range of about 75-125 mg diphenhydramine and a wakefulness promoting agent selected from the group consisting of adrafinil, about 200-350 mg modafinil, about 150-225 mg armodafinil, and about 310-425 mg Caffeine per dosage unit;
a PM dosage for an individual less than 100 lb, comprising a range of about 6-50 mg diphenhydramine per dosage unit;
a PM dosage for an individual of 95-170 lb, comprising a range of about 25-75 mg diphenhydramine per dosage unit;
a PM dosage for an individual of 150-300 lb, comprising a range of about 50-100 mg diphenhydramine per dosage unit; and
a PM dosage for an individual over 270 lb, comprising a range of about 75-125 mg diphenhydramine per dosage unit.

17. The pharmaceutical unit dosage form of claim 16, wherein the dosage form contains release-retarding material; wherein the release-retarding material is a release-retarding matrix, a release-retarding coating, or a combination comprising at least one of the foregoing; wherein the controlled-release formulation provides therapeutically effective plasma levels for greater than about 12 hours after administration at steady state; wherein the release-retarding material is selected from the group consisting of polyethylene glycols, waxes, cellulose derivatives, polyacrylate derivatives, polyvinylpyrrolidine, hydrogenated oils, shellac, zein, fatty acids, gums, and the like.

18. The pharmaceutical unit dosage form of claim 16, wherein the pharmaceutical unit dosage is an oral dosage form comprising an antihistamine, or a pharmaceutically acceptable salt or derivative thereof, and an optional wakefulness promoting agent, or a pharmaceutically acceptable salt or derivative thereof.

19. The pharmaceutical unit dosage form of claim 16, wherein the dosage form is a controlled-release pharmaceutical formulation comprising an antihistamine, or a pharmaceutically acceptable salt or derivative thereof, and a wakefulness promoting agent, or a pharmaceutically acceptable salt or derivative thereof; wherein said controlled release formulation exhibits an in vitro dissolution profile in simulated intestinal fluid medium comprising less than about 70% drug release after 1 hour, at least about 20% drug release after 4 hours, and at least about 30% drug release after 6 hours.

20. The pharmaceutical dosage form according to claim 19 comprises a controlled release component of a wakefulness promoting agent, or a pharmaceutically acceptable salt or derivative thereof and a pharmaceutically acceptable excipient; and an immediately release component of an antihistamine, or a pharmaceutically acceptable salt or derivative thereof; wherein said controlled release component exhibits an in vitro dissolution profile in simulated intestinal fluid medium comprising less than about 70% wakefulness promoting agent release after 1 hour, at least about 20% wakefulness promoting agent release after 4 hours, and at least about 30% wakefulness promoting agent release after 6 hours.

21. The pharmaceutical dosage form according to claim 19 comprises a controlled release component of an antihistamine, or a pharmaceutically acceptable salt or derivative thereof and a pharmaceutically acceptable excipient; and an immediately release component of a wakefulness promoting agent, or a pharmaceutically acceptable salt or derivative thereof; wherein said controlled release component exhibits an in vitro dissolution profile in simulated intestinal fluid medium comprising less than about 70% antihistamine release after 1 hour, at least about 20% antihistamine release after 4 hours, and at least about 30% antihistamine release after 6 hours.
